(19) United States
(12) Patent Application Publication
(10) Pub. No.: US 2008/0221096 A1
(43) Pub. Date: Sep. 11, 2008

(54) Pyrido[3' ,2':4,5]Furo[3,2-d]Pyrimidine Derivatives

(76) Inventors: Joan Taltavull Moll, Barcelona (ES); Luis Miguel Pages Santacana, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/996,781

(22) PCT Filed: Jul. 21, 2006
(86) PCT No.: PCT/EP2006/007218

§ 371 Date: Feb. 14, 2008

(30) Foreign Application Priority Data
Jul. 27, 2005 (ES) P200501840

(51) Int. Cl. A61K 31/5377; C07D 239/70; C07D 295/00; A61P 17/00; A61P 1/00; A61P 11/00; A61K 31/519

(52) U.S. Cl. 514/232.5; 544/247; 514/257; 544/115; 514/232.8; 544/80

(57) ABSTRACT

The present disclosure relates to a pyridofuropyrimidine derivative of formula (I):

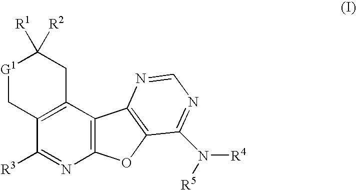

wherein

G¹ is a group chosen from —CR⁶R⁷— and —O— wherein R⁶ and R⁷ are independently chosen from hydrogen atoms and C₁₋₄ alkyl groups; R¹ and R² are independently chosen from hydrogen atoms and C₁₋₄ alkyl groups; R³ is chosen from C₁₋₄ alkyl, C₁₋₄ alkoxy, amino, hydroxy, mono-C₁₋₄alkylamino, di-C₁₋₄alkylamino, C₃₋₈cycloalkylamino, aryl, heteroaryl and saturated N-containing heterocyclyl groups which are bound to the pyridine ring through their nitrogen atom, all of them being optionally substituted by one or more substituents chosen from halogen atoms and hydroxy, C₁₋₄ alkyl, C₁₋₄alkoxy-C₁₋₄alkyl, aryl-C₁₋₄alkyl, —O(CO)OR⁸, C₁₋₄ alkoxy, —(CO)NR⁸R⁹, —CN, —CF₃, —NR⁸R⁹, —SR⁸ and —SO₂NH₂ groups wherein R⁸ and R⁹ are each independently chosen from a hydrogen atom or a C₁₋₄ alkyl group; R⁴ and R⁵ are independently chosen from hydrogen atoms, C₁₋₄alkyl groups, hydroxyl-C₁₋₄alkyl groups and groups of formula (II):

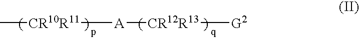

wherein p and q are integers chosen from 0, 1, 2 and 3; A is either a direct bond or a group chosen from —CONR¹⁴—, —NR¹⁴CO—, —O—, —COO—, —OCO—, —S—, —SO— and —SO₂—, wherein each R¹⁰, R¹¹, R¹², R¹³ and R¹⁴ are independently chosen from a hydrogen atom and a C₁₋₄alkyl group and G² is chosen from aryl, heteroaryl and heterocyclyl groups; wherein the group G² is optionally substituted by one or more substituents chosen from halogen atoms and C₁₋₄alkyl, hydroxy, oxo, C₁₋₄alkoxy-C₁₋₄alkyl, aryl-C₁₋₄alkyl, —(CO)OR¹⁶, C₁₋₄alkoxy, —(CO)NR¹⁶R¹⁷, —CN, —CF₃, —NR¹⁶R¹⁷—SR¹⁶ and —SO₂NH₂ groups; wherein R¹⁶ and R¹⁷ each independently chosen from hydrogen atom and a C₁₋₄alkyl group and the pharmaceutically acceptable salts and N-oxides thereof.

The present invention relates to new therapeutically useful pyridofuropyrimidine derivatives, to processes for their preparation and to pharmaceutical compositions containing them. These compounds are potent and selective inhibitors of phosphodiesterase 4 (PDE4) and are thus useful in the treatment, prevention or suppression of pathological conditions, diseases and disorders known to be susceptible of being improved by inhibition of PDE4.

Phosphodiesterases (PDEs) comprise a superfamily of enzymes responsible for the hydrolysis and inactivation of the second messengers cyclic adenosine monophosphate (cAMP) and cyclic guanosine monophosphate (cGMP). Eleven different PDE families have been identified to date (PDE1 to PDE11) which differ in substrate preference, catalytic activity, sensitivity to endogenous activators and inhibitors, and encoding genes.

The PDE4 isoenzyme family exhibits a high affinity for cyclic AMP but has weak affinity for cyclic GMP. Increased cyclic AMP levels caused by PDE4 inhibition are associated with the suppression of cell activation in a wide range of inflammatory and immune cells, including lymphocytes, macrophages, basophils, neutrophils, and eosinophils. Moreover, PDE4 inhibition decreases the release of the cytokine Tumor Necrosis Factor α (TNFα). The biology of PDE4 is described in several recent reviews, for example M. D. Houslay, Prog. Nucleic Acid Res. Mol. Biol. 2001, 69, 249-315; J. E. Souness et al. Immunopharmacol. 2000 47, 127-162; or M. Conti and S. L. Jin, Prog. Nucleic Acid Res. Mol. Biol. 1999, 63, 1-38.

In view of these physiological effects, PDE4 inhibitors of varied chemical structures have been recentlty disclosed for the treatment or prevention of chronic and acute inflammatory diseases and of other pathological conditions, diseases and disorders known to be susceptible to amelioration by inhibition of PDE4. See, for example, U.S. Pat. No. 5,449,686, U.S. Pat. No. 5,710,170, WO 98/45268, WO 99/06404, WO 01/57025, WO 01/57036, WO 01/46184, WO 97/05105, WO 96/40636, U.S. Pat. No. 5,786,354, U.S. Pat. No. 5,773,467, U.S. Pat. No. 5,753,666, U.S. Pat. No. 5,728,712, U.S. Pat. No. 5,693,659, U.S. Pat. No. 5,679,696, U.S. Pat. No. 5,596,013, U.S. Pat. No. 5,541,219, U.S. Pat. No. 5,508,300, U.S. Pat. No. 5,502,072 or H. J. Dyke and J. G. Montana, Exp. Opin. Invest. Drugs 1999, 8, 1301-1325.

A few compounds having the capacity to selectively inhibit phosphodiesterase 4 are in active development. Examples of these compounds are cipamfylline, arofyline, cilomilast, roflumilast, mesopram and pumafentrine.

We have now found that a novel series of pyrido[3′,2′:4,5]furo[3,2-d]pyrimidine derivatives are potent and selective inhibitors of PDE4 and are therefore useful in the treatment or prevention of these pathological conditions, diseases and disorders, in particular asthma, chronic obstructive pulmonary disease, rheumatoid arthritis, atopic dermatitis, psoriasis or irritable bowel disease.

The compounds of the present invention can also be used in combination with other drugs known to be effective in the treatment of these diseases. For example, they can be used in combination with steroids or immunosuppressive agents, such as cyclosporin A, rapamycin or T-cell receptor blockers. In this case the administration of the compounds allows a reduction of the dosage of the other drugs, thus preventing the appearance of the undesired side effects associated with both steroids and immunosuppressants.

Like other PDE4 inhibitors (see references above) the compounds of the invention can also be used for blocking the ulcerogenic effects induced by a variety of etiological agents, such as antiinflammatory drugs (steroidal or non-steroidal antiinflammatory agents), stress, ammonia, ethanol and concentrated acids. They can be used alone or in combination with antacids and/or antisecretory drugs in the preventive and/or curative treatment of gastrointestinal pathologies like drug-induced ulcers, peptic ulcers, H. Pylori-related ulcers, esophagitis and gastro-esophageal reflux disease.

They can also be used in the treatment of pathological situations where damage to the cells or tissues is produced through conditions like anoxia or the production of an excess of free radicals. Examples of such beneficial effects are the protection of cardiac tissue after coronary artery occlusion or the prolongation of cell and tissue viability when the compounds of the invention are added to preserving solutions intended for storage of transplant organs or fluids such as blood or sperm. They are also of benefit on tissue repair and wound healing.

Accordingly, the present invention provides compounds of formula (I), their use in the manufacture of a medicament for the treatment of diseases susceptible of being improved by inhibition of PDE4; methods of treatment of diseases susceptible to amelioration by inhibition of PDE4, which methods comprise the administration to a subject in need of treatment of the compounds of formula (I) and pharmaceutical composition comprising the compounds of formula (I):

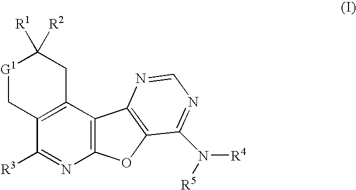

wherein:
G¹ represents a group selected from —CR⁶R⁷— and —O— wherein R⁶ and R⁷ independently represent hydrogen atoms or C₁₋₄ alkyl groups;
R¹ and R² are independently selected from hydrogen atoms and C₁₋₄ alkyl groups;
R³ represents a group selected from C₁₋₄ alkyl, C₁₋₄ alkoxy, amino, hydroxy, mono-C₁₋₄alkylamino, di-C₁₋₄alkylamino, C₃₋₈cycloalkylamino, aryl, heteroaryl and saturated N-containing heterocyclyl groups which are bound to the pyridine ring through their nitrogen atom, all of them being optionally substituted by one or more substituents selected from the group consisting of halogen atoms and hydroxy, C₁₋₄alkyl, C₁₋₄alkoxy-C₁₋₄alkyl, aryl-C₁₋₄alkyl, —O(CO)OR⁸, C₁₋₄alkoxy, —(CO)NR⁸R⁹, —CN, —CF₃, —NR⁸R⁹, —SR¹ and —SO₂NH₂ groups wherein R⁸ and R⁹ each independently represent a hydrogen atom or a C₁₋₄ alkyl group;
R⁴ and R⁵ are independently selected from the group consisting of hydrogen atoms, C₁₋₄alkyl groups, hydroxyl-C₁₋₄alkyl groups and groups of formula (II):

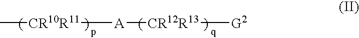

wherein p and q are integers selected from 0, 1, 2 and 3; A is either a direct bond or a group selected from —CONR¹⁴—, —NR¹⁴CO—, —O—, —COO—, —OCO—, —S—, —SO— and —SO₂—, wherein each R¹⁰, R¹¹, R¹², R¹³ and R¹⁴ independently represents a hydrogen atom or a C₁₋₄alkyl group and G² is a group selected from aryl, heteroaryl or heterocyclyl groups; wherein the group G² is optionally substituted by one or more substituents selected from group consisting of halogen atoms and C₁₋₄alkyl, hydroxy, oxo, C₁₋₄alkoxy-C₁₋₄alkyl, aryl-C₁₋₄alkyl, —(CO)OR¹⁶, C₁₋₄alkoxy, —(CO)NR¹⁶R¹⁷, —CN, —CF₃, —NR¹⁶R¹⁷, —SR¹⁶ and —SO₂NH₂ groups; wherein R¹⁶ and R¹⁷ each independently represent a hydrogen atom or a C₁₋₄alkyl group and the pharmaceutically acceptable salts and N-oxides thereof.

Still further objectives of the present invention are to provide processes for preparing said compounds and pharmaceutical compositions comprising an effective amount of said compounds.

As used herein the term alkyl embraces optionally substituted, linear or branched radicals having 1 to 20 carbon atoms or, preferably 1 to 12 carbon atoms. More preferably alkyl radicals are “lower alkyl” radicals having 1 to 8, preferably 1 to 6 and more preferably 1 to 4 carbon atoms.

Examples include methyl, ethyl, n-propyl, i-propyl, n-butyl, sec-butyl, t-butyl, n-pentyl, 1-methylbutyl, 2-methylbutyl, isopentyl, 1-ethylpropyl, 1,1-dimethylpropyl, 1,2-dimethylpropyl, n-hexyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 2-methylpentyl, 3-methylpentyl and iso-hexyl radicals.

When it is mentioned that alkyl radicals may be optionally substituted it is meant to include linear or branched alkyl, alkenyl or alkynyl radicals as defined above, which may be unsubstituted or substituted in any position by one or more substituents, for example by 1, 2 or 3 substituents. When two or more substituents are present, each substituent may be the same or different.

A said optionally substituted alkyl group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, substituents on an alkyl group are themselves unsubstituted. Preferred optionally substituted alkyl groups are unsubstituted or substituted with 1, 2 or 3 fluorine atoms.

As used herein, the term alkoxy (or alkyloxy) embraces optionally substituted, linear or branched oxy-containing radicals each having alkyl portions of 1 to 10 carbon atoms. More preferred alkoxy radicals are “lower alkoxy” radicals having 1 to 8, preferably 1 to 6 and more preferably 1 to 4 carbon atoms.

An alkoxy group is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on an alkoxy group are themselves unsubstituted.

Preferred alkoxy radicals include methoxy, ethoxy, n-propoxy, i-propoxy, n-butoxy, sec-butoxy, t-butoxy, trifluoromethoxy, difluoromethoxy, hydroxymethoxy, 2-hydroxyethoxy and 2-hydroxypropoxy.

As used herein, the term monoalkylamino embraces radicals containing an optionally substituted, linear or branched alkyl radicals of 1 to 10 carbon atoms attached to a divalent —NH— radical. More preferred monoalkylamino radicals are “lower monoalkylamino” radicals having 1 to 8, preferably 1 to 6 and more preferably 1 to 4 carbon atoms.

A monoalkylamino group typically contains an alkyl group which is unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substitutents on a monoalkylamino group are themselves unsubstituted.

Preferred optionally substituted monoalkylamino radicals include methylamino, ethylamino, n-propylamino, i-propylamino, n-butylamino, sec-butylamino, t-butylamino, trifluoromethylamino, difluoromethylamino, hydroxymethylamino, 2-hydroxyethylamino and 2-hydroxypropylamino.

As used herein, the term dialkylamino embraces radicals containing a trivalent nitrogen atoms with two optionally substituted, linear or branched alkyl radicals of 1 to 10 carbon atoms attached thereto. More preferred dialkylamino radicals are “lower dialkylamino” radicals having 1 to 8, preferably 1 to 6 and more preferably 1 to 4 carbon atoms in each alkyl radical.

A dialkylamino group typically contains two alkyl groups, each of which is unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a dialkylamino group are themselves unsubstituted.

Preferred optionally substituted dialkylamino radicals include dimethylamino, diethylamino, methyl(ethyl)amino, di(n-propyl)amino, n-propyl(methyl)amino, n-propyl(ethyl)amino, di(i-propyl)amino, i-propyl(methyl)amino, i-propyl(ethyl)amino, di(n-butyl)amino, n-butyl(methyl)amino, n-butyl(ethyl)amino, n-butyl(i-propyl)amino, di(sec-butyl)amino, sec-butyl(methyl)amino, sec-butyl(ethyl)amino, sec-butyl(n-propyl)amino, sec-butyl(i-propyl)amino, di(t-butyl)amino, t-butyl(methyl)amino, t-butyl(ethyl)amino, t-butyl(n-propyl)amino, t-butyl(i-propyl)amino, trifluoromethyl(methyl)amino, trifluoromethyl(ethyl)amino, trifluoromethyl(n-propyl)amino, trifluoromethyl(i-propyl)amino, trifluoromethyl(n-butyl)amino, trifluoromethyl(sec-butyl)amino, difluoromethyl(methyl)amino, difluoromethyl(ethyl)amino, difluoromethyl(n-propyl)amino, difluoromethyl(i-propyl)amino, difluoromethyl(n-butyl))amino, difluoromethyl(sec-butyl)amino, difluoromethyl(t-butyl)amino, difluoromethyl(trifluoromethyl)amino, hydroxymethyl(methyl)amino, ethyl(hydroxymethyl)amino, hydroxymethyl(n-propyl)amino, hydroxymethyl(i-propyl)amino, n-butyl(hydroxymethyl)amino, sec-butyl(hydroxymethyl)amino, t-butyl(hydroxymethyl)amino, difluoromethyl(hydroxymethyl)amino, hydroxymethyl(trifluoromethyl)amino, hydroxyethyl(methyl)amino, ethyl(hydroxyethyl)amino, hydroxyethyl(n-propyl)amino, hydroxyethyl(i-propyl)amino, n-butyl(hydroxyethyl)amino, sec-butyl(hydroxyethyl)amino, t-butyl(hydroxyethyl)amino, difluoromethyl(hydroxyethyl)amino, hydroxyethyl(trifluoromethyl)amino, hydroxypropyl(methyl)amino, ethyl(hydroxypropyl)amino, hydroxypropyl(n-propyl)amino, hydroxypropyl(i-propyl)amino, n-butyl(hydroxypropyl)amino, sec-butyl(hydroxypropyl)amino, t-butyl(hydroxypropyl)amino, difluoromethyl(hydroxypropyl)amino, hydroxypropyl(trifluoromethyl)amino.

As used herein, the term aryl radical embraces typically a C₅-C₁₄ monocyclic or polycyclic aryl radical such as phenyl, naphthyl, anthranyl and phenanthryl. Phenyl is preferred.

A said optionally substituted aryl radical is typically unsubstituted or substituted with 1, 2 or 3 substituents which may be the same or different. The substituents are preferably selected from halogen atoms, preferably fluorine atoms, hydroxy groups, alkoxycarbonyl groups in which the alkyl moiety has from 1 to 4 carbon atoms, hydroxycarbonyl groups, carbamoyl groups, nitro groups, cyano groups, C₁-C₄ alkyl groups, C₁-C₄ alkoxy groups and C₁-C₄ hydroxyalkyl groups. When an aryl radical carries 2 or more substituents, the substituents may be the same or different. Unless otherwise specified, the substituents on an aryl group are typically themselves unsubstituted.

As used herein, the term heteroaryl radical embraces typically a 5- to 14-membered ring system, preferably a 5- to 10-membered ring system, comprising at least one heteroaromatic ring and containing at least one heteroatom selected from O, S and N. A heteroaryl radical may be a single ring or two or more fused rings wherein at least one ring contains a heteroatom.

A said optionally substituted heteroaryl radical is typically unsubstituted or substituted with 1, 2 or 3 substituents which may be the same or different. The substituents are preferably selected from halogen atoms, preferably fluorine, chlorine or bromine atoms, alkoxycarbonyl groups in which the alkyl moiety has from 1 to 4 carbon atoms, nitro groups, hydroxy groups, C₁-C₄ alkyl groups and C₁-C₄ alkoxy groups. When an heteroaryl radical carries 2 or more substituents, the substituents may be the same or different. Unless otherwise specified, the substituents on a heteroaryl radical are typically themselves unsubstituted.

Examples include pyridyl, pyrazinyl, pyrimidinyl, pyridazinyl, furyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-d]pyrimidinyl, 1H-pyrazolo[3,4-d]pyrimidinyl, thieno[2,3-d]pyrimidnyl and the various pyrrolopyridyl radicals.

Oxadiazolyl, oxazolyl, pyridyl, pyrrolyl, imidazolyl, thiazolyl, thiadiazolyl, thienyl, furanyl, quinolinyl, isoquinolinyl, indolyl, benzoxazolyl, naphthyridinyl, benzofuranyl, pyrazinyl, pyrimidinyl and the various pyrrolopyridyl radicals are preferred.

As used herein, the term heterocyclyl radical embraces typically a non-aromatic, saturated or unsaturated C₃-C₁₀ carbocyclic ring, such as a 5, 6 or 7 membered radical, in which one or more, for example 1, 2, 3 or 4 of the carbon atoms preferably 1 or 2 of the carbon atoms are replaced by a heteroatom selected from N, O and S. Saturated heterocyclyl radicals are preferred. A heterocyclic radical may be a single ring or two or more fused rings wherein at least one ring contains a heteroatom. When a heterocyclyl radical carries 2 or more substituents, the substituents may be the same or different. A N-containing heterocyclyl radical is an heterocyclyl radical in which at least one carbon atom of the carbocyclyl ring is replaced by a nitrogen atom.

A said optionally substituted heterocyclyl radical is typically unsubstituted or substituted with 1, 2 or 3 substituents which may be the same or different. The substituents are preferably selected from halogen atoms, preferably fluorine atoms, hydroxy groups and alkoxy groups having from 1 to 4 carbon atoms. Typically, the substituents on a heterocyclyl radical are themselves unsubstituted.

Examples of heterocyclic radicals include piperidyl, pyrrolidyl, pyrrolinyl, piperazinyl, morpholinyl, thiomorpholinyl, pyrrolyl, pyrazolinyl, pirazolidinyl, quinuclidinyl, triazolyl, pyrazolyl, tetrazolyl, cromanyl, isocromanyl, imidazolidinyl, imidazolyi, oxiranyl, azaridinyl, 4,5-dihydro-oxazolyl and 3-aza-tetrahydrofuranyl. Preferred heterocyclyl radicals are selected from piperidyl, pyrrolidyl, piperazinyl, morpholinyl and thiomorpholinyl.

Where a heterocyclyl radical carries 2 or more substituents, the substituents may be the same or different.

As used herein, some of the atoms, radicals, moieties, chains and cycles present in the general structures of the invention are “optionally substituted”. This means that these atoms, radicals, moieties, chains and cycles can be either unsubstituted or substituted in any position by one or more, for example 1, 2, 3 or 4, substituents, whereby the hydrogen atoms bound to the unsubstituted atoms, radicals, moieties, chains and cycles are replaced by chemically acceptable atoms, radicals, moieties, chains and cycles. When two or more substituents are present, each substituent may be the same or different. The substituents are typically themselves unsubstituted.

As used herein, the term halogen atom embraces chlorine, fluorine, bromine and iodine atoms. A halogen atom is typically a fluorine, chlorine or bromine atom, most preferably chlorine or fluorine. The term halo when used as a prefix has the same meaning.

Compounds containing one or more chiral centre may be used in enantiomerically or diastereoisomerically pure form, or in the form of a mixture of isomers.

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

As used herein, an N-oxide is formed from the tertiary basic amines or imines present in the molecule, using a convenient oxidising agent.

In an embodiment of the present invention in the compounds of formula (I) G¹ represents a group selected from —C(CH₃)₂— and —O—.

In another embodiment of the present invention in the compounds of formula (I) R¹ and R² are both methyl groups;

In another embodiment of the present invention in the compounds of formula (I) R³ represents a group selected from C₁₋₄ alkyl, C₁₋₄ alkoxy, hydroxy, mono-C₁₋₄alkylamino, di-C₁₋₄alkylamino, C₃₋₈cycloalkylamino, and saturated N-containing heterocyclyl groups which are bound to the pyridine ring through their nitrogen atom, all of them being optionally substituted by one or more substituents selected from the group consisting of halogen atoms and hydroxyl or C₁₋₄alkyl groups. It is further preferred that R³ represents a group selected from mono-C₁₋₄alkylamino, di-C₁₋₄alkylamino, C₃₋₈cycloalkylamino, and saturated N-containing heterocyclyl groups bound through the nitrogen atom to the pyridine ring, all of them being unsubstituted or substituted by one hydroxyl group.

In another embodiment of the present invention in the compounds of formula (I) R⁴ is selected from the group consisting of hydrogen atoms, 2-hydroxyethyl and 2-morpholin-4-yletyhyl groups. It is further preferred that R⁴ represents a hydrogen atom.

In still another embodiment of the present invention in the compounds of formula (I) R⁵ is selected from the group consisting of hydrogen atoms hydroxyalkyl groups and groups of formula (II):

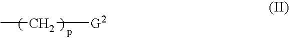

wherein p is an integer selected from 0, 1, 2 and 3; and G² is a group selected from aryl, heteroaryl or heterocyclyl groups which groups are optionally substituted one or more substituents selected from oxo groups and C₁₋₄alkoxy groups. It is preferred that G² is selected from the group consisting of phenyl, pyridine, morpholine and pyrrolidine, optionally substituted with one or more substituents selected from oxo groups and C₁₋₄alkoxy groups

Particular individual compounds of the invention include:
5-Methoxy-2,2-dimethyl-N-(pyridin-3-ylmethyl)-1,2,3,4-tetrahydropyrimido[4′,5′:4,5]furo[2,3-c]isoquinolin-8-amine
2,2-Dimethyl-8-[(pyridin-3-ylmethyl)amino]-1,2,3,4-tetrahydropyrimido[4′,5′:4,5]furo[2,3-c]isoquinolin-5-ol
2,2-Dimethyl-5-morpholin-4-yl-N-(pyridin-3-ylmethyl)-1,2,3,4-tetrahydropyrimido[4′,5′:4,5]furo[2,3-c]isoquinolin-8-amine
2,2-Dimethyl-N-(pyridin-3-ylmethyl)-5-pyrrolidin-1-yl-1,2,3,4-tetrahydropyrimido[4′,5′:4,5]furo[2,3-c]isoquinolin-8-amine
N⁵,N⁵,2,2-Tetramethyl-N8-(pyridin-3-ylmethyl)-1,2,3,4-tetrahydropyrimido[4′,5′:4,5]furo[2,3-c]isoquinoline-5,8-diamine
N⁵-Ethyl-N⁵,2,2-trimethyl-N8-(pyridin-3-ylmethyl)-1,2,3,4-tetrahydropyrimido[4′,5′:4,5]furo[2,3-c]isoquinoline-5,8-diamine
2,2-Dimethyl-5-morpholin-4-yl-N-(2-morpholin-4-ylethyl)-1,4-dihydro-2H-pyrano[4″,3″:4′,5′]pyrido[3′,2′:4,5]furo[3,2-d]pyrimidin-8-amine
2,2-Dimethyl-5-morpholin-4-yl-N-(pyridin-3-ylmethyl)-1,4-dihydro-2H-pyrano[4″,3″:4′,5]pyrido[3′,2′:4,5]furo[3,2-d]pyrimidin-8-amine
N-(2,3-Dimethoxybenzyl)-2,2-dimethyl-5-morpholin-4-yl-1,4-dihydro-2H-pyrano[4″,3″:4′,5]pyrido[3′,2′:4,5]furo[3,2-d]pyrimidin-8-amine
2-[(2,2-Dimethyl-5-morpholin-4-yl-1,4-dihydro-2H-pyrano[4″,3″:4′,5′]pyrido[3′,2′:4,5]furo[3,2-d]pyrimidin-8-yl)(2-morpholin-4-ylethyl)amino]ethanol
N⁵, N⁵,2,2-Tetramethyl-N⁸-(2-morpholin-4-ylethyl)-1,4-dihydro-2H-pyrano[4″,3″:4′,5′]pyrido[3′,2′:4,5]furo[3,2-d]pyrimidine-5,8-diamine
N⁵, N⁵,2,2-Tetramethyl-N⁸-(pyridin-3-ylmethyl)-1,4-dihydro-2H-pyrano[4″,3″:4′,5′]pyrido[3′,2′:4,5]furo[3,2-d]pyrimidine-5,8-diamine
N⁸-(2,3-Dimethoxybenzyl)-N⁵,N⁵,2,2-tetramethyl-1,4-dihydro-2H-pyrano[4″,3″:4′,5′]pyrido[3′,2′:4,5]furo[3,2-d]pyrimidine-5,8-diamine
2,2-Dimethyl-5-morpholin-4-yl-N-(2-morpholin-4-ylethyl)-N-(pyridin-3-ylmethyl)-1,4-dihydro-2H-pyrano[4″,3″:4′,5′]pyrido[3′,2′:4,5]furo[3,2-d]pyrimidin-8-amine
N⁵, N⁵,2,2-Tetramethyl-N⁸-(2-morpholin-4-ylethyl)-N⁸-(pyridin-3-ylmethyl)-1,4-dihydro-2H-pyrano[4″,3″:4′,5′]pyrido[3′,2′:4,5]furo[3,2-d]pyrimidine-5,8-diamine
N⁸-(3,4-Dimethoxybenzyl)-N⁵,N⁵,2,2-tetramethyl-1,4-dihydro-2H-pyrano[4″,3″:4′,5′]pyrido[3′,2′:4,5]furo[3,2-d]pyrimidine-5,8-diamine
5-Methoxy-2,2-dimethyl-N-(2-morpholin-4-ylethyl)-1,2,3,4-tetrahydropyrimido[4′,5′:4,5]furo[2,3-c]isoquinolin-8-amine
1-{3-[(2,2-Dimethyl-5-morpholin-4-yl-1,2,3,4-tetrahydropyrimido[4′,5′:4,5]furo[2,3-c]isoquinolin-8-yl)amino]propyl}pyrrolidin-2-one
2,2-Dimethyl-8-[(2-morpholin-4-ylethyl)amino]-1,2,3,4-tetrahydropyrimido[4′,5′:4,5]furo[2,3-c]isoquinolin-5-ol
2-[(2,2-Dimethyl-5-morpholin-4-yl-1,2,3,4-tetrahydropyrimido[4′,5′:4,5]furo[2,3-c]isoquinolin-8-yl)(2-morpholin-4-ylethyl)amino]ethanol
1-{3-[(5-Methoxy-2,2-dimethyl-1,2,3,4-tetrahydropyrimido[4′,5′:4,5]furo[2,3-c]isoquinolin-8-yl)amino]propyl}pyrrolidin-2-one
N-(2,3-dimethoxybenzyl)-2,2-dimethyl-5-morpholin-4-yl-1,2,3,4-tetrahydropyrimido[4′,5′:4,5]furo[2,3-c]isoquinolin-8-amine
2,2-Dimethyl-5-morpholin-4-yl-N-(2-morpholin-4-ylethyl)-1,2,3,4-tetrahydropyrimido[4′,5′:4,5]furo[2,3-c]isoquinolin-8-amine
2,2-Dimethyl-N-(2-morpholin-4-ylethyl)-5-pyrrolidin-1-yl-1,2,3,4-tetrahydropyrimido[4′,5′:4,5]furo[2,3-c]isoquinolin-8-amine
N⁵,N⁵,2,2-Tetramethyl-N⁸-(2-morpholin-4-ylethyl)-1,2,3,4-tetrahydropyrimido[4′,5′:4,5]furo[2,3-c]isoquinoline-5,8-diamine
2-({2,2-Dimethyl-8-[(2-morpholin-4-ylethyl)amino]-1,2,3,4-tetrahydropyrimido[4′,5′:4,5]furo[2,3-c]isoquinolin-5-yl}amino)ethanol
2,2-Dimethyl-N,N′-bis(2-morpholin-4-ylethyl)-1,2,3,4-tetrahydropyrimido[4′,5′:4,5]furo[2,3-c]isoquinoline-5,8-diamine
1-(3-{[5-(Dimethylamino)-2,2-dimethyl-1,2,3,4-tetrahydropyrimido[4′,5′:4,5]furo[2,3-c]isoquinolin-8-yl]amino}propyl)pyrrolidin-2-one
1-{3-[(2,2-Dimethyl-5-pyrrolidin-1-yl-1,2,3,4-tetrahydropyrimido[4′,5′:4,5]furo[2,3-c]isoquinolin-8-yl)amino]propyl}pyrrolidin-2-one
N⁵-Ethyl-2,2-dimethyl-N⁸-(2-morpholin-4-ylethyl)-1,2,3,4-tetrahydropyrimido[4′,5′:4,5]furo[2,3-c]isoquinoline-5,8-diamine
N⁵-Ethyl-N⁵,2,2-trimethyl-N⁸-(2-morpholin-4-ylethyl)-1,2,3,4-tetrahydropyrimido[4′,5′:4,5]furo[2,3-c]isoquinoline-5,8-diamine
N⁵-Isopropyl-2,2-dimethyl-N⁸-(2-morpholin-4-ylethyl)-1,2,3,4-tetrahydropyrimido[4′,5′:4,5]furo[2,3-c]isoquinoline-5,8-diamine
1-[3-({5-[(2-Hydroxyethyl)amino]-2,2-dimethyl-1,2,3,4-tetrahydropyrimido[4′,5′:4,5]furo[2,3-c]isoquinolin-8-yl}amino)propyl]pyrrolidin-2-one
1-(3-{[5-(Ethylamino)-2,2-dimethyl-1,2,3,4-tetrahydropyrimido[4′,5′:4,5]furo[2,3-c]isoquinolin-8-yl]amino}propyl)pyrrolidin-2-one
1-[3-({5-[Ethyl(methyl)amino]-2,2-dimethyl-1,2,3,4-tetrahydropyrimido[4′,5′:4,5]furo[2,3-c]isoquinolin-8-yl}amino)propyl]pyrrolidin-2-one
2-[[5-(Dimethylamino)-2,2-dimethyl-1,4-dihydro-2H-pyrano[4″,3″:4′,5′]pyrido[3′,2′:4,5]furo[3,2-d]pyrimidin-8-yl](2-morpholin-4-ylethyl)amino]ethanol
1-(3-{[5-(Isopropylamino)-2,2-dimethyl-1,2,3,4-tetrahydropyrimido[4′,5′:4,5]furo[2,3-c]isoquinolin-8-yl]amino}propyl)pyrrolidin-2-one
2-[(5-Methoxy-2,2-dimethyl-1,2,3,4-tetrahydropyrimido[4′,5′:4,5]furo[2,3-c]isoquinolin-8-yl)(2-morpholin-4-ylethyl)amino]ethanol
8-[(2-Hydroxyethyl)(2-morpholin-4-ylethyl)amino]-2,2-dimethyl-1,2,3,4-tetrahydropyrimido[4′,5′:4,5]furo[2,3-c]isoquinolin-5-ol
N⁵-Cyclopropyl-2,2-dimethyl-N⁸-(2-morpholin-4-ylethyl)-1,2,3,4-tetrahydropyrimido[4′,5′:4,5]furo[2,3-c]isoquinoline-5,8-diamine
N⁵-Cyclopentyl-2,2-dimethyl-N-(2-morpholin-4-ylethyl)-1,2,3,4-tetrahydropyrimido[4′,5′:4,5]furo[2,3-c]isoquinoline-5,8-diamine
N⁵-Ethyl-2,2-dimethyl-N⁸-(pyridin-3-ylmethyl)-1,2,3,4-tetrahydropyrimido[4′,5′:4,5]furo[2,3-c]isoquinoline-5,8-diamine
N⁵-Isopropyl-2,2-dimethyl-N⁸-(pyridin-3-ylmethyl)-1,2,3,4-tetrahydropyrimido[4′,5′:4,5]furo[2,3-c]isoquinoline-5,8-diamine
2-({2,2-Dimethyl-8-[(pyridin-3-ylmethyl)amino]-1,2,3,4-tetrahydropyrimido[4′,5′:4,5]furo[2,3-c]isoquinolin-5-yl}amino)ethanol
N⁵-Cyclobutyl-2,2-dimethyl-N⁸-(2-morpholin-4-ylethyl)-1,2,3,4-tetrahydropyrimido[4′,5′:4,5]furo[2,3-c]isoquinoline-5,8-diamine
1-(3-{[2,2-Dimethyl-5-(methylamino)-1,2,3,4-tetrahydropyrimido[4′,5′:4,5]furo[2,3-c]isoquinolin-8-yl]amino}propyl)pyrrolidin-2-one
2,2-Dimethyl-8-{[3-(2-oxopyrrolidin-1-yl)propyl]amino}-2,3,4,6-tetrahydropyrimido[4′,5′:4,5]furo[2,3-c]isoquinolin-5(1H)-one
N⁵,2,2-trimethyl-N⁸-(2-morpholin-4-ylethyl)-1,4-dihydro-2H-pyrano[4″,3″:4′,5′]pyrido[3′,2′:4,5]furo[3,2-d]pyrimidine-5,8-diamine

Of outstanding interest are:
2,2-Dimethyl-5-morpholin-4-yl-N-(pyridin-3-ylmethyl)-1,2,3,4-tetrahydropyrimido[4′,5′:4,5]furo[2,3-c]isoquinolin-8-amine
2,2-Dimethyl-5-morpholin-4-yl-N-(2-morpholin-4-ylethyl)-N-(pyridin-3-ylmethyl)-1,4-dihydro-2H-pyrano[4″,3″:4′,5′]pyrido[3′,2′:4,5]furo[3,2-d]pyrimidin-8-amine
2-[(2,2-Dimethyl-5-morpholin-4-yl-1,2,3,4-tetrahydropyrimido[4′,5′:4,5]furo[2,3-c]isoquinolin-8-yl)(2-morpholin-4-ylethyl)amino]ethanol
2,2-Dimethyl-5-morpholin-4-yl-N-(2-morpholin-4-ylethyl)-1,2,3,4-tetrahydropyrimido[4′,5′:4,5]furo[2,3-c]isoquinolin-8-amine
N⁵-Ethyl-2,2-dimethyl-N⁸-(2-morpholin-4-ylethyl)-1,2,3,4-tetrahydropyrimido[4′,5′:4,5]furo[2,3-c]isoquinoline-5,8-diamine
N⁵-Isopropyl-2,2-dimethyl-N⁸-(2-morpholin-4-ylethyl)-1,2,3,4-tetrahydropyrimido[4′,5′:4,5]furo[2,3-c]isoquinoline-5,8-diamine
1-(3-{[5-(Ethylamino)-2,2-dimethyl-1,2,3,4-tetrahydropyrimido[4′,5′:4,5]furo[2,3-c]isoquinolin-8-yl]amino}propyl)pyrrolidin-2-one
1-(3-{[5-(Isopropylamino)-2,2-dimethyl-1,2,3,4-tetrahydropyrimido[4′,5′:4,5]furo[2,3-c]isoquinolin-8-yl]amino}propyl)pyrrolidin-2-one
N⁵-Isopropyl-2,2-dimethyl-N⁸-(pyridin-3-ylmethyl)-1,2,3,4-tetrahydropyrimido[4′,5′:4,5]furo[2,3-c]isoquinoline-5,8-diamine
N⁵-Cyclobutyl-2,2-dimethyl-N⁸-(2-morpholin-4-ylethyl)-1,2,3,4-tetrahydropyrimido[4′,5′:4,5]furo[2,3-c]isoquinoline-5,8-diamine
   and pharmaceutically acceptable salts thereof.

It is also an objective of the present invention a pharmaceutical composition comprising a compound of formula (I) as hereinabove defined in admixture with a pharmaceutically acceptable diluent or carrier.

It is another object of the present invention a method for treating a subject afflicted with a pathological condition or disease susceptible to amelioration by inhibition of phosphodiesterase 4, which method comprises administering to the said subject an effective amount of a compound of formula (I) as hereinabove defined. It is of particular relevance the method when applied to the treatment of a disease selected from asthma, chronic obstructive pulmonary disease, rheumatoid arthritis, atopic dermatitis, psoriasis or irritable bowel disease.

It is also an object of the present invention to provide a combination product comprising:
(i) a compound of formula (I) as hereinabove defined; and
(ii) another compound selected from (a) steroids, (b) immunosuppressive agents, (c) T-cell receptor blockers and (d) antiinflammatory drugs for simultaneous, separate or sequential use in the treatment of the human or animal body.

It is also an embodiment of the present invention a compound of formula (I) as hereinabove defined for use as a medicament. The compound may be used in the preparation of a medicament for the treatment of diseases or disorders susceptible to amelioration by inhibition of phosphodiesterase 4, in particular a disease selected from the group consisting of asthma, chronic obstructive pulmonary disease, rheumatoid arthritis, atopic dermatitis, psoriasis or irritable bowel disease.

According to a further feature of the present invention, the compounds of formula (I) may be prepared by one of the processes described below.

Compounds Ia wherein R³ is a monosubstituted, disubstituted or unsubstituted amino group may be obtained as shown in Scheme 1.

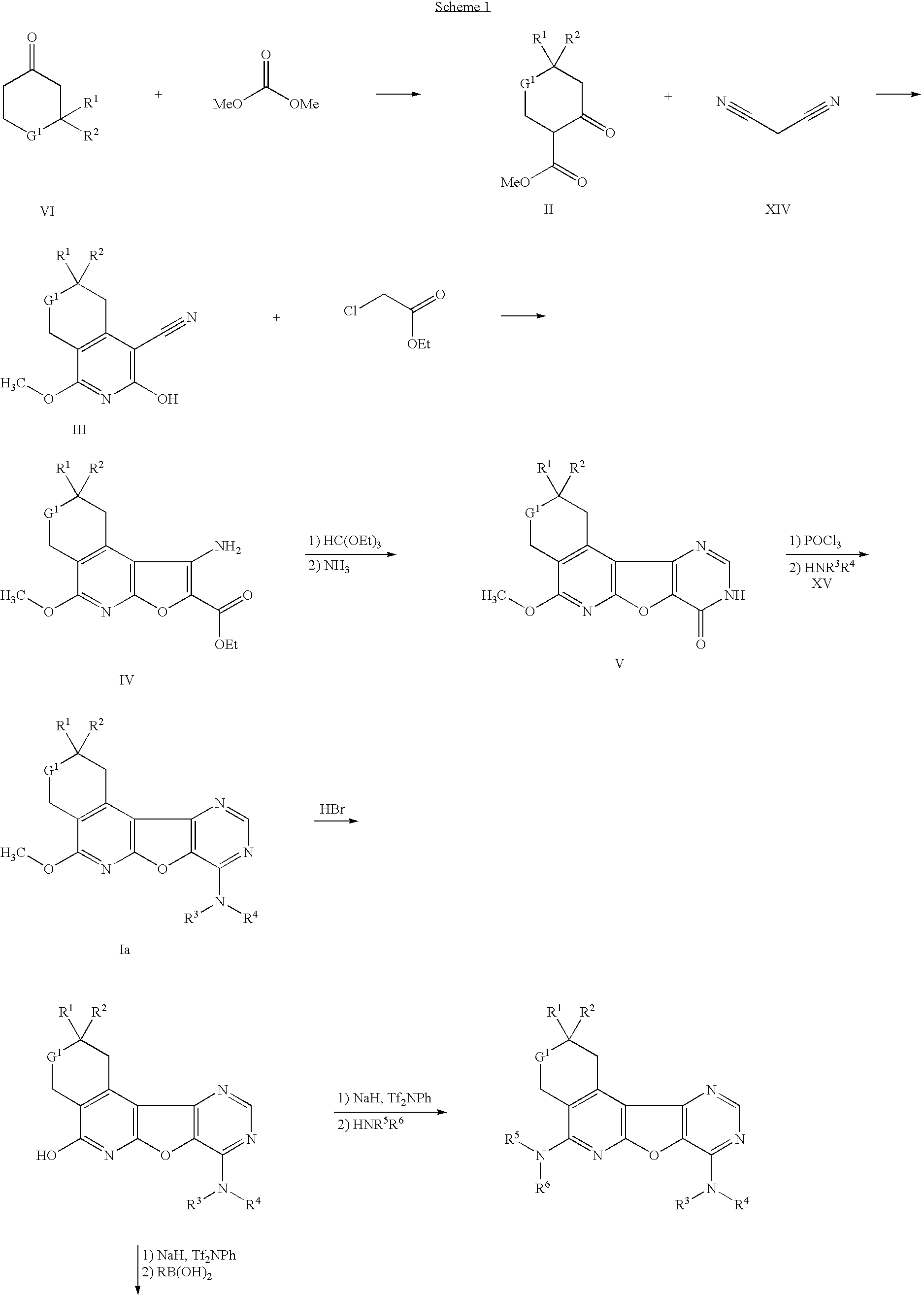

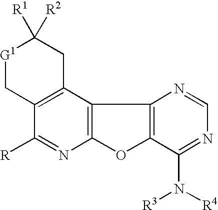

A ketone of formula VI, wherein G¹, R¹ and R² are as hereinbefore defined, is reacted with dialkylcarbonate, preferably dimethyl carbonate in the presence of sodium hydride to yield the heterocycle of formula II, according to the method described by L. A. Paquette at J. Org. Chem., 1991, 56, 6199. Ketones VI are commercially available or prepared according to the methods described at C. Ainsworth Org. Synth., 1959, 39, 536, J. Cologne, A. Varagnat Bull. Soc. Chim. France, 1964, 10, 2499-504, and E. M. Kosower, T. S. Sorensen, 1963, 28, 687.

Reaction of compound II with malononitrile XIV yields the pyridine derivative III, as described by J. L. van der Baan et al at Tetrahedron, 1974, 30, 2447-53.

Subsequent cyclocondensation of compound III with ethyl 2-chloroacetate in the presence of a base such as potassium carbonate yields the furopyridine compound IV, according to C. Peinador et al J. Het. Chem., 1992, 29, 1693 or C. Peinador et al Bioorg. Med. Chem., 1998, 6, 1911.

The pyridothienopyrimidine derivative V is synthesized by cyclisation of intermediate IV with triethyl orthoformate and ammonia, as described at C. Peinador et al Bioorg. Med. Chem., 1998, 6, 1911. The reaction can be carried out in a solvent, preferably a polar aprotic solvent, such as N,N-dimethylformamide, dioxane, acetone or tetrahydrofuran, in the presence of an organic base, preferably an amine base, such as triethylamine and at a temperature from 15° C. to 40° C. The reaction can also be carried out in the absence of a solvent.

The corresponding chloroimine derivative of V is synthesized using phosphorous oxychloride as solvent, and the resulting intermediate is reacted with an amine of formula XV, wherein R³ and R⁴ are as hereinbefore defined, to give the compound Ia.

Compound Ia is demethylated by heating it at 100° C. in bromhydric acid, and the resulting hydroxypyridine Ib leads to the desired final compound Ic through the intermediate triflate, which is substituted with the appropriate amine HNR⁵R⁶ or, alternatively, with an alkyl, aryl or heteroaryl through the corresponding boronate using the suitable catalyst

When the defined groups R′, R″ and R¹ to R⁶ are susceptible to chemical reaction under the conditions of the hereinbefore described processes or are incompatible with said processes, conventional protecting groups may be used in accordance with standard practice, for example see T. W. Greene and P. G. M. Wuts in ‘Protective Groups in Organic Chemistry’, 3^{rd} Edition, John Wiley & Sons (1999). It may be that deprotection will form the last step in the synthesis of compounds of formula I.

According to an embodiment of the present invention, the pyridothienopyrimidine derivatives of general formula (Ic) are prepared by the process described below and shown in Scheme 2.

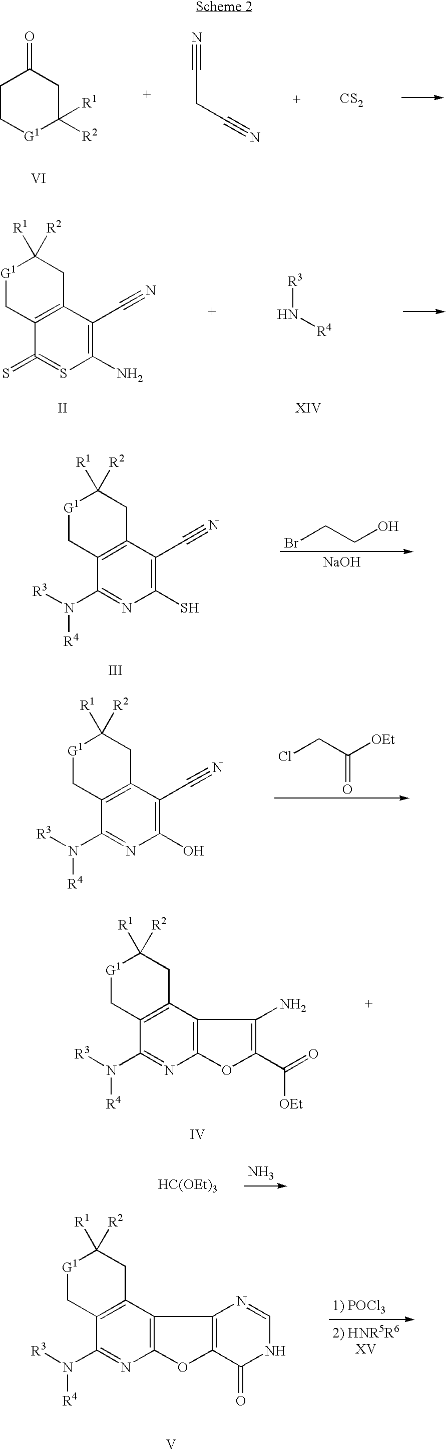

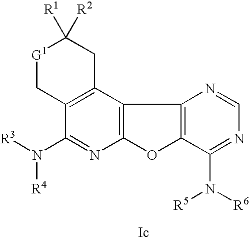

A ketone of formula VI, wherein G¹, R¹ and R² are as hereinbefore defined, is condensed with malononitrile in the presence of carbon disulfide to yield the heterocycle of formula II, according to the method described by E. G. Paronikyan and A. S. Noravyan at Chem. Heterocycl. Compd (NY), 1999, 35(7), 799-803. Ketones VI are commercially available or prepared according to the methods described at C. Ainsworth Org. Synth., 1959, 39, 536, J. Cologne, A. Varagnat Bull. Soc. Chim. France, 1964, 10, 2499-504, and E. M. Kosower, T. S. Sorensen, 1963, 28, 687.

Reaction of compound II with an amine HNR³R⁴ of formula XIV, wherein R³ and R⁴ are as hereinbefore defined, yields the pyridine derivative III, as described by K. Gewald et al at J. Prakt. Chem., 1973, 315(4), 679-689.

Compound III is converted to the corresponding hydroxypyridine by heating it with 2-bromoethanol in basic conditions.

Subsequent cyclocondensation of compound III derivative with ethyl 2-chloroacetate in the presence of a base such as potassium carbonate yields the furopyridine compound IV, according to C. Peinador et al J. Het. Chem., 1992, 29, 1693 or C. Peinador et al Bioorg. Med. Chem., 1998, 6, 1911.

The pyridofuropyrimidine derivative V is synthesized by cyclisation of intermediate IV with triethylorthoformate. The reaction can be carried out in a solvent, preferably a polar aprotic solvent, such as N,N-dimethylformamide, dioxane, acetone or tetrahydrofuran, in the presence of an organic base, preferably an amine base, such as triethylamine and at a temperature from 15° C. to 40° C. The reaction can also be carried out in the absence of a solvent.

The corresponding chloroimine derivative of V is synthesized using phosphorous oxychloride as solvent, and the resulting intermediate is reacted with an amine of formula XV, wherein R⁴ and R⁵ are as hereinbefore defined, to give the desired final compound Ic.

The pharmaceutically acceptable salts of the compounds of the present invention represented by formula Ia, Ib and Ic may be acid addition salts or alkali addition salts. Examples of the acid addition salts include mineral acid addition salts such as, for example, hydrochloride, hydrobromide, hydroiodide, sulfate, nitrate, phosphate, and organic acid addition salts such as, for example, acetate, maleate, fumarate, citrate, oxalate, succinate, tartrate, malate, mandelate, methanesulfonate, and p-toluenesulfonate. Examples of the alkali addition salts include inorganic salts such as, for example sodium, potassium, calcium and ammonium salts and organic alkali salts such as, for example, ethylenediamine, ethanolamine, N,N-dialkylenethanolamine, triethanolamine and basic amino acid salts.

The compounds of the present invention represented by the above described formula (Ia, Ib and Ic) may include enantiomers depending on their asymmetry or diastereoisomers. The single isomers and mixtures of the isomers fall within the scope of the present invention.

The compounds of formulae VI, XIV, XV and XVI are known compounds or can be prepared by analogy with known methods.

### Pharmacological Activity

### PDE4 Assay Procedure

Compounds to be tested were resuspended in DMSO at a stock concentration of 1 mM. The compounds were tested at different concentrations varying from 10 μM to 10 μM to calculate an IC₅₀. These dilutions were done in 96-well plates. In some cases, plates containing diluted compounds were frozen before being assayed. In these cases, the plates were thawed at room temperature and stirred for 15 minutes.

Ten microliters of the diluted compounds were poured into a “low binding” assay plate. Eighty microliters of reaction mixture containing 50 mM Tris pH 7.5, 8.3 mM MgCl₂, 1.7 mM EGTA, and 15 nM [3H]-cAMP were added to each well. The reaction was initiated by adding ten microliters of a solution containing PDE4. The plate was then incubated under stirring for 1 hour at room temperature. After incubation the reaction was stopped with 50 microlitres of SPA beads, and the reaction was allowed to incubate for another 20 minutes at room temperature before measuring radioactivity using standard instrumentation.

The reaction mixture was prepared by adding 90 ml of H₂O to 10 ml of 10× assay buffer (500 mM Tris pH 7.5, 83 mM MgCl₂, 17 mM EGTA), and 40 microlitres 1 μCi/μL [3H]-cAMP. SPA beads solution was prepared by adding 500 mg to 28 ml H₂O for a final concentration of 20 mg/ml beads and 18 mM zinc sulphate.

The results are shown in Table 1.

TABLE 1

| Example | IC₅₀ PDE4 (nM) |
|---|---|
| 3 | 5.3 |
| 14 | 10.0 |
| 20 | 0.8 |
| 23 | 8.9 |
| 31 | 2.0 |
| 33 | 0.2 |
| 35 | 1.6 |
| 38 | 0.2 |
| 44 | 0.2 |
| 46 | 0.7 |

It can be seen from Table 1 that the compounds of formula (I) are potent inhibitors of phosphodiesterase 4 (PDE 4). Preferred pyridofuropyrimidine derivatives of the invention possess an IC₅₀ value for the inhibition of PDE4 (determined as defined above) of less than 100 nM, preferably less than 50 nM and most preferably less than 30 nM.

The compounds are also capable of blocking the production of some pro-inflammatory cytokines such as, for example, TNFα. Thus, they can be used in the treatment of allergic, inflammatory and immunological diseases, as well as those diseases or conditions where the blockade of pro-inflammatory cytokines or the selective inhibition of PDE 4 could be of benefit.

These disease states include asthma, chronic obstructive pulmonary disease, allergic rhinitis, rheumatoid arthritis, osteoarthritis, osteoporosis, bone-formation disorders, glomerulonephritis, multiple sclerosis, ankylosing spondylitis, Graves ophtalmopathy, myasthenia gravis, diabetes insipidus, graft rejection, gastrointestinal disorders such as ulcerative colitis or Crohn disease, septic shock, adult distress respiratory syndrome, and skin diseases such as atopic dermatitis, contact dermatitis, acute dermatomyositis and psoriasis. They can also be used as improvers of cerebrovascular function as well as in the treatment of other CNS related diseases such as dementia, Alzheimer's disease, depression, and as nootropic agents.

The compounds of the present invention are also of benefit when administered in combination with other drugs such as steroids and immunosuppressive agents, such as cyclosporin A, rapamycin or T-cell receptor blockers. In this case the administration of the compounds allows a reduction of the dosage of the other drugs, thus preventing the appearance of the undesired side effects associated with both steroids and immunosuppressants. The compounds of the invention have also shown their efficacy in blocking, after preventive and/or curative treatment, the erosive and ulcerogenic effects induced by a variety of etiological agents, such as antiinflammatory drugs (steroidal or non-steroidal antiinflammatory agents), stress, ammonia, ethanol and concentrated acids.

They can be used alone or in combination with antacids and/or antisecretory drugs in the preventive and/or curative treatment of gastrointestinal pathologies like drug-induced ulcers, peptic ulcers, H. Pylori-related ulcers, esophagitis and gastro-esophageal reflux disease. They can also be used in the treatment of pathological situations where damage to the cells or tissues is produced through conditions like anoxia or the production of an excess of free radicals. Examples of such beneficial effects are the protection of cardiac tissue after coronary artery occlusion or the prolongation of cell and tissue viability when the compounds of the invention are added to preserving solutions intended for storage of transplant organs or fluids such as blood or sperm. They are also of benefit on tissue repair and wound healing.

Accordingly, the pyridofuropyrimidine derivatives of the invention and pharmaceutically acceptable salts thereof, and pharmaceutical compositions comprising such compound and/or salts thereof, may be used in a method of treatment of disorders of the human body which comprises administering to a patient requiring such treatment an effective amount of a pyridothienopyrimidine derivative of the invention or a pharmaceutically acceptable salt thereof.

The present invention also provides pharmaceutical compositions which comprise, as an active ingredient, at least a pyridothienopyrimidine derivative of formula (I) or a pharmaceutically acceptable salt thereof in association with a pharmaceutically acceptable excipient such as a carrier or diluent. The active ingredient may comprise 0.001% to 99% by weight, preferably 0.01% to 90% by weight, of the composition depending upon the nature of the formulation and whether further dilution is to be made prior to application. Preferably the compositions are made up in a form suitable for oral, topical, nasal, rectal, percutaneous or injectable administration.

The pharmaceutically acceptable excipients which are admixed with the active compound, or salts of such compound, to form the compositions of this invention are well-known per se and the actual excipients used depend inter alia on the intended method of administering the compositions.

Compositions for oral administration may take the form of tablets, retard tablets, sublingual tablets, capsules, inhalation aerosols, inhalation solutions, dry powder inhalation, or liquid preparations, such as mixtures, elixirs, syrups or suspensions, all containing the compound of the invention; such preparations may be made by methods well-known in the art.

The diluents which may be used in the preparation of the compositions include those liquid and solid diluents which are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Compositions for topical administration may take the form of ointments, creams or lotions, all containing the compound of the invention; such preparations may be made by methods well-known in the art.

Effective doses are normally in the range of 10-600 mg of active ingredient per day. Daily dosage may be administered in one or more treatments, preferably from 1 to 4 treatments, per day.

The syntheses of the compounds of the invention and of the intermediates for use therein are illustrated by the following Examples (including Preparation Examples (Preparations 1 to 63)) which do not limit the scope of the invention in any way.

¹H Nuclear Magnetic Resonance Spectra were recorded on a Varian Gemini 300 spectrometer.

Low Resolution Mass Spectra (m/z) were recorded on a Micromass ZMD mass spectrometer using ESI ionization.

Melting points were recorded using a Perkin Elmer DSC-7 apparatus.

The chromatographic separations were obtained using a Waters 2690 system equipped with a Symmetry C18 (2.1×10 mm, 3.5 mM) column. The mobile phase was formic acid (0.4 mL), ammonia (0.1 mL), methanol (500 mL) and acetonitrile (500 mL) (B) and formic acid (0.46 mL), ammonia (0.115 mL) and water (1000 mL) (A): initially from 0% to 95% of B in 20 min, and then 4 min. with 95% of B. The reequilibration time between two injections was 5 min. The flow rate was 0.4 mL/min. The injection volume was 5 microliter. Diode array chromatograms were collected at 210 nM.

### PREPARATION EXAMPLES

### Preparation 1

### 3,3-Dimethylcyclohexanone

To a suspension of copper(I) cyanide (2.46 g, 27.5 mmol) cooled at 0° C. a 3.0M solution of methyl magnesium bromide (18.25 ml, 54.8 mmol) is dropwise added. Once the addition is completed, the reaction mixture is stirred for 30 min more at 0° C. and then cooled to −78° C. A solution of 3-methyl-2-cyclohexen-1-one (1.0 g, 9.07 mmol) in ethyl ether (15 ml) is then dropwise added. When the addition is over, the reaction mixture is stirred between −40° C. and −20° C. for two hours. Finally, an aqueous solution of phosphate buffer (pH=7.2, 90 ml) is carefully added to quench the reaction, followed by saturated solution of ammonium chloride (35 ml). The system is allowed to reach room temperature and the two phases separated. The aqueous phase is extracted twice with ethyl ether and the organic phases washed with brine, dried over magnesium sulphate, filtered and the solvents evaporated under vacuum. 1.08 g of the desired final compound, as an orange oil, is obtained, pure enough so as to be used in the next synthetic step without further purification. Yield=94%

¹H NMR (200 MHz, CDCl₃) δ ppm 0.98 (s, 6H) 1.59 (m, 2H) 1.89 (m, 2H) 2.16 (s, 2H) 2.28 (t, J=6.62 Hz, 2H).

### Preparation 2

### Methyl 4,4-dimethyl-2-oxocyclohexancarboxylate

Sodium hydride (60%,1.95 g, 81.2 mmol) is suspended in THF (120 ml), dimethyl carbonate (17 ml, 198.0 mmol) is added and the mixture is heated to reflux. 3,3-Dimethylcyclohexanone (5.0 g, 39.6 mmol, see Preparation 1) in THF (60 ml) is dropwise added and this mixture is refluxed for 2 h. Once at room temperature, the reaction mixture is poured on saturated solution of ammonium chloride (125 ml). After successive extractions with ethyl ether, the organic phase is washed with water and brine, dried over magnesium sulfate, filtered and the solvent evaporated under reduced pressure. 5.94 g of the final compound are obtained as an oil, pure enough to perform the next synthetic step. Yield=81%.

¹H NMR (300 MHz, CHLOROFORM-D) δ ppm 1.0 (s, 6H) 1.4 (t, J=6.6 Hz, 2H) 2.1 (s, 2H) 2.2 (m, 3H) 3.8 (s, 3H).

### Preparation 3

### 3-Hydroxy-1-methoxy-6,6-dimethyl-5,6,7,8-tetrahydroisoquinolin-4-carbonitrile

Methyl 4,4-dimethyl-2-oxocyclohexancarboxylate (8.4 g, 45.5 mmol, see Preparation 2) is dissolved in a 1:1 mixture toluene/methanol (2×30 ml) and ammonium acetate (0.5 g, 6.8 mmol) and acetic acid (2.8 ml) are added. The reaction mixture is refluxed overnight and the solvent evaporated under reduced pressure. 1N NaOH (120 ml) is added to the residue and the precipitated solid is filtered and washed with water. Once dried, 6.9 g of the final compound are obtained. Yield=66%.

¹H NMR (300 MHz, CHLOROFORM-D) δ ppm 0.9 (s, 6H) 1.4 (t, J=6.6 Hz, 2H) 2.2 (s, 2H) 2.3 (t, J=6.6 Hz, 2H) 3.7 (s, 3H).

### Preparation 4

### Ethyl (4-cyano-1-methoxy-6,6-dimethyl-5,6,7,8-tetrahydroisoquinolin-3-yloxy)acetate

3-Hydroxy-1-methoxy-6,6-dimethyl-5,6,7,8-tetrahydroisoquinolin-4-carbonitrile (6.9 g, 29.8 mmol, see Preparation 3) is dissolved in acetone (180 ml) and potassium carbonate (9.9 g, 71.6 mmol) is added. After dropwise addition of ethyl bromoacetate (3.3 ml, 29.8 mmol) at room temperature, this mixture is refluxed under nitrogen for 3 h. The solvent is evaporated under reduced pressure and the residue is redissolved in water/Et₂O. After usual work-up, 8.4 g of the desired final molecule are obtained as an oil. Yield=89%.

¹H NMR (300 MHz, CHLOROFORM-D) δ ppm 1.0 (s, 6H) 1.3 (t, J=6.6 Hz, 3H) 1.5 (t, J=6.6 Hz, 2H) 2.5 (t, J=6.6 Hz, 2H) 2.6 (s, 2H) 3.9 (s, 3H) 4.2 (q, J=6.6 Hz, 2H) 4.9 (s, 2H).

### Preparation 5

### Ethyl 1-amino-5-methoxy-8,8-dimethyl-6,7,8,9-tetrahydrofuro[2,3-c]isoquinolin-2-carboxylate

Ethyl (4-cyano-1-methoxy-6,6-dimethyl-5,6,7,8-tetrahydroisoquinolin-3-yloxy)acetate (17.2 g, 54 mmol, see Preparation 4) is dissolved in ethanol (350 ml) and sodium ethoxide (17.5 ml, 54 mmol of a 21 wt. % solution in denaturated ethyl alcohol) is added. After 8 h of reflux, the solvent is evaporated under reduced pressure and the residue is partitioned between chloroform and saturated solution of ammonium chloride. The organic phase is separated and the aqueous phase is extracted twice with chloroform. The organic phase is washed with brine and dried over magnesium sulfate. Once the solvent is evaporated, 15.1 g of the final compound are obtained as a pale pink solid, pure enough to perform the next synthetic step.

Yield=88%.

¹H NMR (300 MHz, CHLOROFORM-D) δ ppm 1.0 (s, 6H) 1.4 (t, J=6.6 Hz, 3H) 1.6 (t, J=6.6 Hz, 2H) 2.6 (t, J=6.6 Hz, 2H) 2.8 (s, 2H) 4.0 (s, 3H) 4.3 (q, J=6.6 Hz, 2H) 4.9 (bs, 2H).

### Preparation 6

### 5-Methoxy-2,2-dimethyl-1,2,3,4-tetrahydropyrimido[4′,5′:4,5]furo[2,3-c]isoquinolin-8-ol

Ethyl 1-amino-5-methoxy-8,8-dimethyl-6,7,8,9-tetrahydrofuro[2,3-c]isoquinolin-2-carboxylate (1.0 g, 3.3 mmol, see Preparation 5) is refluxed for 6 h in triethyl orthoformate. Then, the solvent is evaporated under reduced pressure and the residue is redissolved in ethanol (15 ml) and concentrated ammonia (12 ml) and heated under reluxed for 18 h. After the solvent has been evaporated, the residue is partitioned between water and ethyl acetate. The organic phase is separated and the aqueous phase is twice extracted with ethyl acetate. The organic phase is washed with water, dried over magnesium sulfate, filtered and the solvent evaporated. 0.82 g of a brownish solid are obtained, which is rinsed with dichloromethane to yield 0.2 g of the final product as a brownish solid. Yield=20%.

¹H NMR (300 MHz, CHLOROFORM-D) δ ppm 1.0 (s, 6H) 1.6 (t, J=6.6 Hz, 2H) 2.6 (t, J=6.6 Hz, 2H) 3.0 (s, 2H) 4.0 (s, 3H) 8.2 (s, 1H).

### Preparation 7

### 8-Chloro-2,2-dimethyl-5-methoxy-1,2,3,4-tetrahydropyrimido[4′,5′:4,5]furo[2,3-c]isoquinoline

5-Methoxy-2,2-dimethyl-1,2,3,4-tetrahydropyrimido[4′,5′:4,5]furo[2,3-c]isoquinolin-8-ol (9.9 g, 32.7 mmol, see Preparation 6) is suspended in phosphorous oxychloride (40 ml) and heated to reflux for 3 h. The solvent is evaporated under reduced pressure and the residue is worked-up as usual with ethyl acetate and water. The reaction crude is purified by silica-gel chromatography, eluting with CH₂Cl₂/MeOH 99:1, to yield 5.2 g of the desired final product. Yield=50%.

¹H NMR (300 MHz, CHLOROFORM-D) δ ppm 1.0 (s, 6H) 1.6 (t, J=6.6 Hz, 2H) 2.6 (t, J=6.6 Hz, 2H) 3.0 (s, 2H) 4.0 (s, 3H) 8.9 (s, 1H).

### Preparation 8

### 1-Hydroxy-5-methyl-hexa-1,4-dien-3-one

To a suspension of sodium hydride (2.04 g, 50.9 mmol) in ethyl ether (100 ml) ethanol (0.25 ml) was added in one portion. Once this suspension is cooled in an ice-bath, a mixture of mesityl oxide (5.0 g, 50.9 mmol) and ethyl formate (6.17 ml, 76.4 mmol) in ethyl ether (20 ml) is dropwise added. This final mixture is stirred at this temperature for 6 h and then allowed to reach room temperature overnight. Ethanol (1 ml) is then added and the reaction mixture is stirred at room temperature for one hour. Water (10 ml) is added in one portion and two phases are separated. The organic phase is washed twice with water. These aqueous phases are put together and washed with ethyl ether, then acidified with 6N chlorhidric acid (8.25 ml) and finally extracted repeatedly with ethyl ether. The collected organic phases are washed with brine, dried over magnesium sulfate, filtered and the solvent evaporated under vacuum. 5.10 g of the desired compound is obtained as an orange oil, pure enough to perform the next synthetic step. Yield=79%.

¹H NMR (200 MHz, CDCl₃) δ ppm 1.9 (s, 3H), 2.2 (s, 3H), 3.5 (m. 1H), 5.4 (d, 1H), 5.8 (d, 1H), 8.2 (d, 1H).

### Preparation 9

### 2,2-Dimethyl-2,3-dihydropyran-4-one

A suspension of 1-hydroxy-5-methyl-hexa-1,4-dien-3-one (0.5 g, 3.96 mmol, see Preparation 8), mercurium sulphate (0.05 g, 0.17 mmol) and 10% sulfuric acid (5 ml) is heated at 100° C. for 3 h. The resultant mixture is poured over an ice bath and basified with 2N NaOH to pH=11. After extraction with ethyl ether, the organic phase is washed with brine, dried over magnesium sulfate, filtered and the solvent evaporated under vacuum to yield 0.2 g of the desired final product. Further extraction with ethyl ether of the acidified aqueous phase yields 0.3 g more of final product. Yield=60%.

¹H NMR (200 MHz, CDCl₃) δ ppm 1.45 (s, 6H), 2.5 (s, 2H), 5.4 (d, 2H), 7.2 (d, 2H).

### Preparation 10

### 2,2-Dimethyltetrahydropyran-4-one

The resulting compound of preparation 9 (0.5 g, 3.96 mmol) is hydrogenated at 30 psi in a Parr apparatus using 10% Pd over charcoal (0.05 g) as catalyst and a mixture of ethyl acetate (10 ml) and acetic acid (0.5 ml) as solvent until the reaction is completed. The catalyst is then filtered and the liquid phase is washed with sodium bicarbonate, water and brine, dried over magnesium sulfate, filtered and the solvent evaporated under vacuum, to yield 0.35 g of the desired final compound as a yellowish oil. Yield=69%.

¹H NMR (200 MHz, CDCl₃) δ ppm 1.3 (s, 6H), 2.4 (s, 2H), 2.45 (t, 2H), 4.05 (t, 2H).

### Preparation 11

### 6-Amino-3,3-dimethyl-8-thioxo-4,8-dihydro-1H,3H-thiopyrano[3,4-c]pyran-5-carbonitrile

2,2-Dimethyltetrahydropyran-4-one (5.0 g, 32.0 mmol, see Preparation 10) is solved in methanol (4.7 ml) and carbon disulfide (4.7 ml, 48.8 mmol) is added in one portion. Malononitrile (2.6 g, 39.0 mmol) is added portionwise and, finally, triethylamine (1.95 ml). The reaction mixture is stirred at room temperature for 48 h. An orange precipitate is formed, which is filtered (3.90 g) and is consistent with the desired compound. From the liquid phase, 0.89 g more of 6-amino-3,3-dimethyl-8-thioxo-4,8-dihydro-1H,3H-thiopyrano[3,4-c]pyran-5-carbonitrile were isolated by flash chromatography, eluting first with CH₂Cl₂ and next with the mixture of solvents CH₂Cl₂:MeOH 98:2. Yield=48%.

¹H NMR (200 MHz, CDCl₃) δ ppm 1.30 (s, 6H), 2.62 (s, 2H), 4.66 (s, 2H), 7.91 (s, 2H).

### Preparation 12

### 6-Mercapto-3,3-dimethyl-8-morpholin-4-yl-3,4-dihydro-1H-pyrano[3,4-c]pyridine-5-carbonitrile

The product resulting from preparation 11 (3.9 g, 15.45 mmol) is suspended in ethanol (17 ml) and morpholine (6.7 ml, 77.3 mmol) is added. The reaction mixture is refluxed under nitrogen overnight. Then the system is allowed to reach room temperature and the reaction mixture is left in an ice bath for two hours. The solid formed is filtrated and washed twice with ethanol. After drying, 3.12 g of the final compound are obtained as a dark solid, pure enough to perform the next step. Yield=66%.

¹H NMR (200 MHz, CDCl₃) δ ppm 1.30 (s, 6H), 2.75 (s, 2H), 3.3 (m, 4H), 3.75 (m, 4H), 4.5 (s, 2H).

### Preparation 13

### 3,3-Dimethyl-8-morpholin-4-yl-6-oxo-3,4,6,7-tetrahydro-1H-pyrano[3,4-c]pyridine-5-carbonitrile

6-Mercapto-3,3-dimethyl-8-morpholin-4-yl-3,4-dihydro-1H-pyrano[3,4-c]pyridine-5-carbonitrile (2.8 g, 7.2 mmol, see Preparation 12) is suspended in a mixture of NaOH 1N (7.2 ml) and methanol (20 ml) and 2-bromoethanol (513 μl, 7.2 mmol) is added. This reaction mixture is stirred overnight and methanol is evaporated under reduced pressure. The residue is resuspended in NaOH 1N (55 ml), ethanol (55 ml) and methylglycol (55 ml) and heated at 135° C. during 5 h. The reaction solution is acidified to pH=2 with HCl 2N and extracted with dichloromethane. The organic phase is washed with water and brine, dried over MgSO₄ and evaporated under reduced pressure. 1.8 g of the final compound is obtained as a solid. Yield=88%

¹H NMR (300 MHz, DMSO-D6) δ ppm 1.2 (s, 6H) 2.5 (m, 2H) 2.7 (s, 2H) 3.2 (m, 4H) 3.7 (m, 4H) 4.4 (m, 2H).

### Preparation 14

### Ethyl 2-(5-cyano-3,3-dimethyl-8-morpholin-4-yl-3,4-dihydro-1H-pyrano[3,4-c]pyridin-6-yloxy)acetate

3,3-Dimethyl-8-morpholin-4-yl-6-oxo-3,4,6,7-tetrahydro-1H-pyrano[3,4-c]pyridine-5-carbonitrile (1.9 g, 6.6 mmol, see Preparation 13) is dissolved in acetone (35 ml). Potassium carbonate (908 mg, 6.6 mmol) and ethyl 2-bromoacetate (726 μl, 6.6 mmol) are added and the mixture is refluxed overnight. The reaction mixture is poured onto 150 ml of ice-water. The insoluble solid is filtrated and dried. 1.98 g of the final compound are obtained. Yield=80%.

¹H NMR (300 MHz, CHLOROFORM-D) δ ppm 1.3 (t, J=7.1 Hz, 3H) 1.3 (s, 6H) 2.8 (s, 2H) 3.2 (m, 4H) 3.8 (m, 4H) 4.2 (q, J=7.1 Hz, 2H) 4.5 (s, 2H) 4.9 (s, 2H).

### Preparation 15

### Ethyl 1-amino-8,8-dimethyl-5-morpholin-4-yl-8,9-dihydro-6H-furo[2,3-b]pyrano[4,3-d]pyridine-2-carboxylate

Ethyl 2-(5-cyano-3,3-dimethyl-8-morpholin-4-yl-3,4-dihydro-1H-pyrano[3,4-c]pyridin-6-yloxy)acetate (1.7 g, 4.6 mmol, see Preparation 14) is suspended in DMF (20 ml) and cesium carbonate is added (3.0 g, 9.3 mmol). This reaction mixture is heated at 120° C. for 4 h. The solvent is evaporated under reduced pressure and the residue is partitioned between water and ethyl acetate. The organic phase is separated and the aqueous phase is extracted twice with ethyl acetate. The organic phase is washed successively with water and brine, dried over magnesium sulfate, filtered and the solvent evaporated under reduced pressure. 980 mg of the final compound are obtained. Yield=56%.

¹H NMR (300 MHz, CHLOROFORM-D) δ ppm 1.4 (m, 9H) 3.0 (s, 2H) 3.2 (m, 4H) 3.8 (m, 4H) 4.4 (q, J=7.1 Hz, 2H) 4.7 (s, 2H) 5.1 (s, 2H).

### Preparation 16

### Ethyl 1-{[(1E)-ethoxymethylene]amino}-8,8-dimethyl-5-morpholin-4-yl-8,9-dihydro-6H-furo[2,3-b]pyrano[4,3-d]pyridine-2-carboxylate

Ethyl 1-amino-8,8-dimethyl-5-morpholin-4-yl-8,9-dihydro-6H-furo[2,3-b]pyrano[4,3-d]pyridine-2-carboxylate (980 mg, 2.6 mmol, see Preparation 15) is suspended in triethyl orthoformate (10 ml) and heated to reflux for 6 h. The solvent is evaporated under reduced pressure and the residue is rinsed with ethanol. The insoluble solid is filtrated, washed with ethyl ether and dried. 650 mg of the final compound are obtained. Yield=58%

¹H NMR (300 MHz, CHLOROFORM-D) δ ppm 1.4 (m, 9H) 1.5 (t, J=7.1 Hz, 3H) 3.0 (s, 2H) 3.2 (m, 4H) 3.8 (m, 4H) 4.3 (q, J=7.1 Hz, 2H) 4.4 (q, J=7.0 Hz, 2H) 4.7 (s, 2H) 7.9 (s, 1H).

### Preparation 17

### 2,2-Dimethyl-5-morpholin-4-yl-1,4-dihydro-2H-pyrano[4″,3″:4′,5′]pyrido[3′,2′:4,5]furo[3,2-d]pyrimidin-8(9H)-one

Ethyl 1-{[(1E)-ethoxymethylene]amino}-8,8-dimethyl-5-morpholin-4-yl-8,9-dihydro-6H-furo[2,3-b]pyrano[4,3-d]pyridine-2-carboxylate (650 mg, 1.5 mmol, see Preparation 16) is suspended in ethanol (10 ml) and concentrated ammonia (8 ml) is added. After refluxing for 5 h, the reaction is over. The reaction mixture is cooled to room temperature and then left overnight at +5° C. The precipitated solid is filtered, washed with ethanol and dried. 454 mg of the final compound are obtained. Yield=85%.

¹H NMR (300 MHz, DMSO-D6) δ ppm 1.3 (s, 6H) 3.2 (m, J=8.2 Hz, 6H) 3.8 (m, 4H) 4.7 (s, 2H) 8.2 (s, 1H).

### Preparation 18

### 8-Chloro-2,2-dimethyl-5-morpholin-4-yl-1,4-dihydro-2H-pyrano[4″,3″:4′,5′]pyrido[3′,2′:4,5]furo[3,2-d]pyrimidine

2,2-Dimethyl-5-morpholin-4-yl-1,4-dihydro-2H-pyrano[4″,3″:4′,5′]pyrido[3′,2′:4,5]furo[3,2-d]pyrimidin-8(9H)-one (454 mg, 1.3 mmol, see Preparation 17) is suspended in phosphorous oxychloride (2 ml) and heated to reflux for 90 min. The solvent is evaporated under reduced pressure and the residue is worked-up with water and chloroform as usual. 460 mg of the final compound as a solid are obtained. Yield=96%.

¹H NMR (300 MHz, CHLOROFORM-D) δ ppm 1.4 (s, 6H) 3.4 (s, 2H) 3.4 (m, 4H) 3.9 (m, 4H) 4.8 (s, 2H) 8.9 (s, 1H).

### Preparation 19

### 6-Mercapto-3,3-dimethyl-8-dimethylamino-3,4-dihydro-1H-pyrano[3,4-c]pyridine-5-carbonitrile

The product resulting from preparation 11 (5.0 g, 19.9 mmol) is suspended in ethanol (5 ml) and dimethylamine (5.6M solution in ethanol, 20.2 ml, 113 mmol) is added. The reaction mixture is heated at 85° C. in a sealed tube under nitrogen overnight. Then the system is allowed to reach room temperature and the solvent is evaporated under reduced pressure. The residue is passed through a silica-gel column eluting first with CH₂Cl₂/MeOH 98:2 and then with CH₂Cl₂/MeOH 95:5.1.9 g of the final compound are obtained. Yield=36%.

¹H NMR (300 MHz, CHLOROFORM-D) δ ppm 1.3 (s, 6H) 2.7 (s, 2H) 3.0 (s, 6H) 4.6 (s, 2H).

### Preparation 20

### 3,3-Dimethyl-8-dimethylamino-6-oxo-3,4,6,7-tetrahydro-1H-pyrano[3,4-c]pyridine-5-carbonitrile

6-Mercapto-3,3-dimethyl-8-dimethylamino-3,4-dihydro-1H-pyrano[3,4-c]pyridine-5-carbonitrile (1.9 g, 7.2 mmol, see Preparation 19) is suspended in a mixture of NaOH 1N (7.2 ml) and methanol (20 ml) and 2-bromoethanol (511 μl, 7.2 mmol) is added. This reaction mixture is stirred overnight and methanol is evaporated under reduced pressure. The residue is resuspended in NaOH 1N (55 ml), ethanol (55 ml) and methylglycol (55 ml) and heated at 135° C. during 5 h. The reaction solution is acidified to pH=2 with HCl 2N and extracted with chloroform. The organic phase is washed with water and brine, dried over MgSO₄ and evaporated under reduced pressure. 1.8 g of the final compound is obtained as a solid. Yield=100%.

¹H NMR (300 MHz, CHLOROFORM-D) δ ppm 1.3 (s, 6H) 2.7 (s, 2H) 3.1 (s, 6H) 4.5 (s, 2H).

### Preparation 21

### Ethyl 2-(5-cyano-3,3-dimethyl-8-dimethylamino-3,4-dihydro-1H-pyrano[3,4-c]pyridin-6-yloxy)acetate

3,3-Dimethyl-8-dimethylamino-6-oxo-3,4,6,7-tetrahydro-1H-pyrano[3,4-c]pyridine-5-carbonitrile (1.8 g, 7.2 mmol, see Preparation 20) is dissolved in acetone (35 ml). Potassium carbonate (995 mg, 7.2 mmol) and ethyl 2-bromoacetate (796 μl, 7.2 mmol) are added and the mixture is refluxed overnight. The reaction mixture is poured onto 150 ml of ice-water. The insoluble solid is filtrated and dried. 1.8 g of the final compound are obtained. Yield=75%.

¹H NMR (300 MHz, CHLOROFORM-D) δ ppm 1.2 (t, 3H) 1.3 (s, 6H) 2.8 (s, 2H) 3.0 (s, 6H) 4.2 (q, 2H) 4.6 (s, 2H) 4.9 (s, 2H).

### Preparation 22

### Ethyl 1-amino-8,8-dimethyl-5-dimethylamino-8,9-dihydro-6H-furo[2,3-b]pyrano[4,3-d]pyridine-2-carboxylate

Ethyl 2-(5-cyano-3,3-dimethyl-8-dimethylamino-3,4-dihydro-1H-pyrano[3,4-c]pyridin-6-yloxy)acetate (1.8 g, 5.4 mmol, see Preparation 21) is suspended in DMF (40 ml) and cesium carbonate is added (3.5 g, 10.8 mmol). This reaction mixture is heated at 120° C. for 3 h. The solvent is evaporated under reduced pressure and the residue is partitioned between water and chloroform. The aqueous phase is neutralised with HCl 2N and extracted three times with chloroform. The organic phase is washed successively with water and brine, dried over magnesium sulfate, filtered and the solvent evaporated under reduced pressure. The reaction crude is passed through a silica-gel column eluting first with dichloromethane and then with CH₂Cl₂/MeOH 98:2.1.3 g of the final compound are obtained. Yield=74%.

¹H NMR (300 MHz, CHLOROFORM-D) δ ppm 1.4 (m, 9H) 2.9 (s, 6H) 3.0 (s, 2H) 4.4 (q, 2H) 4.7 (s, 2H) 5.1 (bs, 2H).

### Preparation 23

### Ethyl 1-{[(1E)-ethoxymethylene]amino}-8,8-dimethyl-5-dimethylamino-8,9-dihydro-6H-furo[2,3-b]pyrano[4,3-d]pyridine-2-carboxylate

Ethyl 1-amino-8,8-dimethyl-5-dimethylamino-8,9-dihydro-6H-furo[2,3-b]pyrano[4,3-d]pyridine-2-carboxylate (1.3 g, 4.0 mmol, see Preparation 22) is suspended in triethyl orthoformate (15 ml) and heated to reflux for 6 h. The solvent is evaporated under reduced pressure and the residue is pure enough to perform the next synthetic step (see Preparation 24).

LRMS: m/z 390 (M+1)⁺.

### Preparation 24

### 2,2-Dimethyl-5-dimethylamino-1,4-dihydro-2H-pyrano[4″,3″:4′,5′]pyrido[3′,2′:4,5]furo[3,2-d]pyrimidin-8(9H)-one

Ethyl 1-{[(1E)-ethoxymethylene]amino}-8,8-dimethyl-5-dimethylamino-8,9-dihydro-6H-furo[2,3-b]pyrano[4,3-d]pyridine-2-carboxylate (1.6 g, 4.0 mmol, see Preparation 23) is suspended in ethanol (20 ml) and concentrated ammonia (16 ml) is added. After refluxing for 5 h, the reaction is over. The reaction mixture is cooled to room temperature and then left overnight at +5° C. As no precipitate is observed, the solvent is evaporated under pressure. The residue is worked-up with ethyl acetate and water. 1.0 g of the final compound is obtained. Yield=85%.

¹H NMR (300 MHz, DMSO-D6) δ ppm 1.3 (s, 6H) 3.0 (s, 6H) 3.2 (s, 2H) 4.7 (s, 2H) 8.2 (s, 1H).

### Preparation 25

### 8-Chloro-2,2-dimethyl-5-dimethylamino-1,4-dihydro-2H-pyrano[4″,3″:4′,5′]pyrido[3′,2′:4,5]furo[3,2-d]pyrimidine

2,2-Dimethyl-5-dimethylamino-1,4-dihydro-2H-pyrano[4″,3″:4′,5′]pyrido[3′,2′:4,5]furo[3,2-d]pyrimidin-8(9H)-one (1.0 g, 3.4 mmol, see Preparation 24) is suspended in phosphorous oxychloride (10 ml) and heated to reflux for 2 h. The solvent is evaporated under reduced pressure and the residue is worked-up with water and chloroform as usual. The residue is passed through a silica-gel column eluting with CH₂Cl₂/MeOH 98:2 to yield 736 mg of the final compound as a solid. Yield=65%.

¹H NMR (300 MHz, CHLOROFORM-D) δ ppm 1.4 (s, 6H) 3.1 (s, 6H) 3.3 (s, 2H) 4.8 (s, 2H) 8.9 (s, 1H).

### Preparation 26

### 3-Amino-6,6-dimethyl-1-thioxo-5,6,7,8-tetrahydro-1H-isothiochromene-4-carbonitrile

2,2-Dimethylcyclohexanone (1.15 g, 9.07 mmol, see Preparation 1) is solved in methanol (1.10 ml) and carbon disulfide (1.10 ml, 18.2 mmol) is added in one portion. Malononitrile (0.60 g, 9.07 mmol) is added portionwise and, finally, triethylamine is added (0.44 ml). The reaction mixture is stirred at room temperature for 48 h. The solvent is evaporated under vacuum and 0.84 g of 2-(3,3-dimethylcyclohexylidene)malononitrile were isolated by flash chromatography, eluting first with CH₂Cl₂ and next with the mixture of solvents. This intermediate compound was solved in methanol (0.56 ml) and carbon disulfide (2 equivalents) and triethylamine (0.35 eq.) were added. After 48 h stirring at room temperature, a solid is filtered and washed with methanol. It weighs 0.45 g and its ¹HNMR is consistent with the final product. From the methanolic phase, another 0.5 g of the final compound were isolated by flash chromatography, eluting with CH₂Cl₂:MeOH 95:5. Global yield=42%.

¹H NMR (200 MHz, CDCl₃) δ ppm 1.01 (s, 6H) 1.57 (m, 2H) 2.52 (s, 2H) 2.76 (t, J=6.62 Hz, 2H) 5.67 (s, 2H).

### Preparation 27

### 3-Mercapto-6,6-dimethyl-1-morpholin-4-yl-5,6,7,8-tetrahydroisoquinoline-4-carbonitrile

The product resulting from preparation 26 (0.94 g, 3.75 mmol) is suspended in ethanol (4.5 ml) and morpholine (1.86 ml, 21.4 mmol) is added. The reaction mixture is refluxed under nitrogen overnight. Then the system is allowed to reach room temperature and the reaction mixture is left in an ice bath for two hours. The solid formed is filtrated and washed twice with ethanol. After drying, 0.35 g of the final compound are obtained as a dark solid, pure enough to perform the next step. Yield=31%.

¹H NMR (200 MHz, CDCl₃) δ ppm 1.01 (s, 6H) 1.5 (t, J=6.99 Hz, 2H) 2.2 (m, 1H) 2.47 (t, J=6.99, 2H) 2.6 (s, 2H) 3.3 (m, 4H) 3.9 (m, 4H).

### Preparation 28

### 6,6-Dimethyl-1-morpholin-4-yl-3-oxo-2,3,5,6,7,8-hexahydroisoquinolin-4-carbonitrile

3-Mercapto-6,6-dimethyl-1-morpholin-4-yl-5,6,7,8-tetrahydroisoquinoline-4-carbonitrile (3.0 g, 9.8 mmol, see Preparation 27) is suspended in a mixture of NaOH 1N (9.8 ml) and methanol (30 ml) and 2-bromoethanol (691 μl, 9.8 mmol) is added. This reaction mixture is stirred overnight and methanol is evaporated under reduced pressure. The residue is resuspended in NaOH 1N (75 ml), ethanol (75 ml) and methylglycol (75 ml) and heated at 135° C. during 5 h. The reaction solution is neutralised with HCl 2N and extracted with chloroform. The organic phase is washed with water and brine, dried over MgSO₄ and evaporated under reduced pressure. 2.7 g of the final compound are obtained as a solid. Yield=97%.

LRMS: m/z 288 (M+1)⁺.

### Preparation 29

### Ethyl (4-cyano-6,6-dimethyl-1-morpholin-4-yl-5,6,7,8-tetrahydroisoquinolin-3-yloxy)acetate

6,6-Dimethyl-1-morpholin-4-yl-3-oxo-2,3,5,6,7,8-hexahydroisoquinolin-4-carbonitrile (2.7 g, 9.4 mmol, see Preparation 28) is dissolved in acetone (55 ml). Potassium carbonate (1.3 mg, 9.4 mmol) and ethyl 2-bromoacetate (1.0 ml, 9.4 mmol) are added and the mixture is refluxed overnight. The solvent is evaporated under reduced pressure and the residue is passed through a silica-gel column eluting first with dichloromethane and then with CH₂Cl₂/MeOH 98:2 to yield 2.7 g of the final compound as a brown solid. Yield=75%.

LRMS: m/z 374 (M+1)⁺.

### Preparation 30

### Ethyl 1-amino-8,8-dimethyl-5-morpholin-4-yl-6,7,8,9-tetrahydrofuro[2,3-c]isoquinoline-2-carboxylate

Ethyl (4-cyano-6,6-dimethyl-1-morpholin-4-yl-5,6,7,8-tetrahydroisoquinolin-3-yloxy)acetate (3.2 g, 8.6 mmol, see Preparation 29) is suspended in DMF (65 ml) and cesium carbonate is added (5.6 g, 17.2 mmol). This reaction mixture is heated at 120° C. for 5 h. The solvent is evaporated under reduced pressure and the residue is partitioned between water and chloroform. The organic phase is separated and the aqueous phase is extracted twice with ethyl acetate. The organic phase is washed successively with water and brine, dried over magnesium sulfate, filtered and the solvent evaporated under reduced pressure. 3.2 g of the final compound are obtained. Yield=100%.

¹H NMR (300 MHz, CHLOROFORM-D) δ ppm 1.0 (s, 6H) 1.3 (t, J=7.1 Hz, 3H) 1.5 (t, 2H) 2.6 (t, 2H) 3.0 (s, 2H) 3.2 (m, 4H) 3.8 (m, 4H) 4.2 (q, J=7.1 Hz, 2H) 4.9 (s, 2H).

### Preparation 31

### 2,2-Dimethyl-5-morpholin-4-yl-1,2,3,4-tetrahydropyrimido[4′,5′:4,5]furo[2,3-c]isoquinolin-8(9H)-one

Ethyl 1-amino-8,8-dimethyl-5-morpholin-4-yl-6,7,8,9-tetrahydrofuro[2,3-c] isoquinoline-2-carboxylate (3.2 g, 8.6 mmol, see Preparation 30) is suspended in triethyl orthoformate (30 ml) and the reaction mixture is refluxed for 6 h. The solvent is evaporated under reduced pressure. The residue is suspended in ethanol (40 ml) and concentrated ammonia (30 ml) and refluxed overnight. The solvent is evaporated under reduced pressure and the residue is partitioned between water and ethyl acetate. The aqueous phase is extracted three times. The organic phase is washed with water and brine, dried over magnesium sulphate, filtered and evaporated. The residue is passed through a silica-gel column eluting with CH₂Cl₂/MeOH 95:5 to yield 899 mg of the final compound as a yellow solid. Yield=75%.

¹H NMR (300 MHz, DMSO-D6) δ ppm 1.1 (s, 6H) 1.7 (t, 2H) 2.8 (t, 2H) 3.2 (s, 2H) 3.4 (m, 4H) 3.9 (m, 4H) 8.2 (s, 1H).

### Preparation 32

### 8-Chloro-2,2-dimethyl-5-morpholin-4-yl-1,2,3,4-tetrahydropyrimido[4′,5′:4,5]furo[2,3-c]isoquinoline

2,2-Dimethyl-5-morpholin-4-yl-1,2,3,4-tetrahydropyrimido[4′,5′:4,5]furo[2,3-c]isoquinolin-8(9H)-one (899 mg, 2.5 mmol, see Preparation 31) is suspended in phosphorous oxychloride (2 ml) and heated to reflux for 2 h. The solvent is evaporated under reduced pressure and the residue is worked-up with water and chloroform as usual. The residue is passed through a silica-gel column eluting with CH₂Cl₂/MeOH 98:2 to yield 290 mg of the final compound as a yellow solid. Yield=75%. 460 mg of the final compound. Yield=31%.

¹H NMR (300 MHz, DMSO-D6) δ ppm 1.10 (s, 3H) 1.15 (s, 3H) 1.7 (t, 2H) 2.8 (t, 2H) 3.2 (s, 2H) 3.4 (m, 4H) 3.9 (m, 4H) 8.15 (s, 1H).

### Preparation 33

### 1-[3-({5-[Benzyl(methyl)amino]-2,2-dimethyl-1,2,3,4-tetrahydropyrimido[4′,5′:4,5]furo[2,3-c]isoquinolin-8-yl}amino)propyl]pyrrolidin-2-one

2,2-Dimethyl-8-{[3-(2-oxopyrrolidin-1-yl)propyl]amino}-2,3,4,6-tetrahydropyrimido[4′,5′:4,5]furo[2,3-c]isoquinolin-5(1H)-one (162 mg, 0.40 mmol, see Example 26) dissolved in THF (2 ml) is dropwise added to a suspension of sodium hydride (60% dispersion in mineral oil, 15.8 mg, 0.40 mmol) in THF (10 ml). Then, N-phenylbistrifluorometansulfonamide (141 mg, 0.40 mmol) is added and the reaction mixture is stirred during one hour. Benzyl(methyl)amine (510 μl, 4.0 mmol) is then added and the reaction is stirred overnight. The solvent is evaporated under reduced pressure and the residue is redissolved in ethyl acetate and water. The organic phase is separated and the aqueous phase is extracted twice with ethyl acetate. The organic phase is washed with water and brine, dried over magnesium sulfate, filtered and evaporated. The residue is purified by flash chromatography, eluting with CH₂Cl₂/MeOH 98:2.130 mg of the final compound are obtained. Yield=64%.

LRMS: m/z 513 (M+1)⁺.

### Preparation 34

### 8-[Benzyl(methyl)amino]-6-mercapto-3,3-dimethyl-3,4-dihydro-1H-pyrano[3,4-c]pyridine-5-carbonitrile

The product resulting from preparation 11 (2.3 g, 9.1 mmol) is suspended in ethanol (8 ml) and benzylmethylamine (7.1 ml, 54.6 mmol) is added. The reaction mixture is heated at 90° C. in a sealed tube under nitrogen during 48 h. Then the system is allowed to reach room temperature and the solvent is evaporated under reduced pressure. The residue is passed through a silica-gel column eluting first with CH₂Cl₂/MeOH 98:2 and then with CH₂Cl₂/MeOH 95:5. 1.2 g of the final compound are obtained. Yield=39%.

¹H NMR (300 MHz, CHLOROFORM-D) δ ppm 1.3 (s, 6H) 1.6 (bs, 1H) 2.8 (s, 2H) 3.0 (s, 3H) 4.5 (s, 2H) 4.6 (s, 2H) 7.4 (m, 5H).

### Preparation 35

### 8-[Benzyl(methyl)amino]-6-hydroxy-3,3-dimethyl-3,4-dihydro-1H-pyrano[3,4-c]pyridine-5-carbonitrile

8-[Benzyl(methyl)amino]-6-mercapto-3,3-dimethyl-3,4-dihydro-1H-pyrano[3,4-c]pyridine-5-carbonitrile (1.0 g, 3.0 mmol, see Preparation 34) is suspended in a mixture of NaOH 1N (3 ml) and methanol (10 ml) and 2-bromoethanol (209 μl, 3 mmol) is added. This reaction mixture is stirred overnight and methanol is evaporated under reduced pressure. The residue is resuspended in NaOH 1N (20 ml), ethanol (20 ml) and methylglycol (20 ml) and heated at 135° C. overnight. The reaction solution is diluted with ethyl acetate, washed with water and brine, dried over MgSO₄ and evaporated under reduced pressure. 0.9 g of the final compound is obtained as a solid and pure enough to perform the next synthetic step. Yield=95%.

¹H NMR (300 MHz, CHLOROFORM-D) δ ppm 1.3 (s, 6H) 2.8 (s, 3H) 3.0 (s, 2H) 4.5 (s, 4H) 7.3 (m, 5H).

### Preparation 36

### Ethyl 2-{8-[benzyl(methyl)amino]-5-cyano-3,3-dimethyl-3,4-dihydro-1H-pyrano[3,4-c]pyridin-6-yloxy}acetate

8-[Benzyl(methyl)amino]-6-hydroxy-3,3-dimethyl-3,4-dihydro-1H-pyrano[3,4-c]pyridine-5-carbonitrile (0.9 g, 2.9 mmol, see Preparation 35) is dissolved in acetone (35 ml). Potassium carbonate (402 mg, 2.9 mmol) and ethyl 2-bromoacetate (321 μl, 2.9 mmol) are added and the mixture is refluxed for 3 h. The solvent is evaporated under reduced pressure and the residue is redissolved in ethyl acetate. This organic phase is washed twice with saturated solution of ammonium chloride, dried over magnesium sulfate, filtered and the solvent evaporated. 1.2 g of the final compound as an oil are obtained, pure enough to perform the next synthetic step. Yield=100%.

¹H NMR (300 MHz, CHLOROFORM-D) δ ppm 1.25 (t, 3H) 1.3 (s, 6H) 2.8 (s, 3H) 2.9 (s, 2H) 4.1 (q, 2H) 4.5 (s, 2H) 4.7 (s, 2H) 4.8 (s, 2H) 7.3 (m, 5H).

### Preparation 37

### Ethyl 1-amino-8,8-dimethyl-5-[benzyl(methyl)amino]-8,9-dihydro-6H-furo[2,3-b]pyrano[4,3-d]pyridine-2-carboxylate

Ethyl 2-{8-[benzyl(methyl)amino]-5-cyano-3,3-dimethyl-3,4-dihydro-1H-pyrano[3,4-c]pyridin-6-yloxy}acetate (1.2 g, 2.9 mmol, see Preparation 36) is suspended in DMF (20 ml) and cesium carbonate is added (1.9 g, 5.8 mmol). This reaction mixture is heated at 120° C. for 4 h. The solvent is evaporated under reduced pressure and the residue is partitioned between saturated solution of ammonium chloride and ethyl acetate. The aqueous phase is extracted three times with ethyl acetate. The organic phase is washed successively with saturated solution of ammonium chloride and brine, dried over magnesium sulfate, filtered and the solvent evaporated under reduced pressure. 861 mg of the final compound as a solid are obtained. Yield=72%.

¹H NMR (300 MHz, CHLOROFORM-D) δ ppm 1.4 (s, 6H) 1.42 (t, 3H) 2.8 (s, 3H) 2.9 (s, 2H) 3.0 (s, 2H) 4.4 (q, 2H) 4.8 (s, 2H) 5.1 (bs, 2H) 7.3 (m, 5H).

### Preparation 38

### 2,2-Dimethyl-5-[benzyl(methyl)amino]-1,4-dihydro-2H-pyrano[4″,3″:4′,5′]pyrido[3′,2′:4,5]furo[3,2-d]pyrimidin-8(9H)-one

Ethyl 1-amino-8,8-dimethyl-5-[benzyl(methyl)amino]-8,9-dihydro-6H-furo[2,3-b]pyrano[4,3-d]pyridine-2-carboxylate (861 mg, 2.1 mmol, see Preparation 37) is suspended in triethyl orthoformate (10 ml) and heated to reflux for 4 h. The solvent is evaporated under reduced pressure and the residue is suspended in ethanol (15 ml) and concentrated ammonia (10 ml) is added. After refluxing for 18 h, the reaction is over. The reaction mixture is cooled to room temperature and then left overnight at +5° C. As no precipitate is observed, the solvent is evaporated under pressure. The residue is worked-up with ethyl acetate and water. 592 mg of the final compound are obtained. Yield=72%.

¹H NMR (300 MHz, CHLOROFORM-D) δ ppm 1.4 (s, 6H) 2.8 (s, 1H) 3.0 (s, 3H) 3.3 (s, 2H) 4.5 (s, 2H) 4.8 (s, 2H) 7.3 (m, 5H) 8.2 (s, 1H).

### Preparation 39

### 8-Chloro-2,2-dimethyl-5-[benzyl(methyl)amino]-1,4-dihydro-2H-pyrano[4″,3″:4′,5′]pyrido[3′,2′:4,5]furo[3,2-d]pyrimidine

2,2-Dimethyl-5-[benzyl(methyl)amino]-1,4-dihydro-2H-pyrano[4″,3″:4′,5′]pyrido[3′,2′:4,5]furo[3,2-d]pyrimidin-8(9H)-one (592 mg, 1.5 mmol, see Preparation 38) is suspended in phosphorous oxychloride (5 ml) and heated to reflux for 2 h. The solvent is evaporated under reduced pressure and the residue is worked-up with water and ethyl acetate as usual. The residue is passed through a silica-gel column eluting with CH₂Cl₂/MeOH 98:2 to yield 321 mg of the final compound as a solid. Yield=52%.

¹H NMR (300 MHz, CHLOROFORM-D) δ ppm 1.4 (s, 6H) 3.0 (s, 3H) 3.4 (s, 2H) 4.6 (s, 2H) 4.8 (s, 2H) 7.3 (m 5H) 8.9 (s, 1H).

### Preparation 40

### N⁵-Benzyl-N⁵,2,2-trimethyl-N⁸-(2-morpholin-4-ylethyl)-1,4-dihydro-2H-pyrano[4″,3″:4′,5′]pyrido[3′,2′:4,5]furo[3,2-d]pyrimidine-5,8-diamine

8-Chloro-2,2-dimethyl-5-[benzyl(methyl)amino]-1,4-dihydro-2H-pyrano[4″,3″:4′,5′]pyrido[3′,2′:4,5]furo[3,2-d]pyrimidine (150 mg, 0.4 mmol, see Preparation 39) is suspended in ethanol (5 ml) and 2-morpholine-4-ylethylamine (240 μl, 0.9 mmol) is added. The reaction mixture is heated at 85° C. for 48 h. The solvent is evaporated under reduced pressure and the residue is passed through a silica-gel column eluting dichloromethane first and then successively with the mixtures CH₂Cl₂/MeOH 99:1 and 98:2.118 mg of the final compound are obtained. Yield=64%.

LRMS: m/z 502 (M+1)⁺.

### EXAMPLES

### Example 1

### 5-Methoxy-2,2-dimethyl-N-(pyridin-3-ylmethyl)-1,2,3,4-tetrahydropyrimido[4′,5′:4,5]furo[2,3-c]isoquinolin-8-amine

8-Chloro-2,2-dimethyl-5-methoxy-1,2,3,4-tetrahydropyrimido[4′,5′:4,5]furo[2,3-c]isoquinoline (60.0 mg, 0.2 mmol, see Preparation 7) is suspended in ethanol (5 ml) and pyridin-3-ylmethylamine (0.1 ml, 1.1 mmol) is added. The reaction mixture is refluxed two days. The solvent is evaporated under reduced pressure and the residue is passed through a silica-gel column, eluting with CH₂Cl₂/MeOH 99:1. 60 mg of the final desired product are obtained as solid. Yield=81%

¹H NMR (300 MHz, CHLOROFORM-D) δ ppm 1.1 (s, 6H) 1.7 (t, J=6.6 Hz, 2H) 2.7 (t, J=6.6 Hz, 2H) 3.2 (s, 2H) 4.1 (s, 3H) 4.90 (d, J=6.5, 2H) 5.5 (t, J=6.6 Hz, 1H) 7.3 (m, 1H) 7.7 (m, 1H) 8.6 (m, 1H) 8.7 (m, 2H).

### Example 2

### 2,2-Dimethyl-8-[(pyridin-3-ylmethyl)amino]-1,2,3,4-tetrahydropyrimido[4′,5′:4,5]furo[2,3-c]isoquinolin-5-ol

5-Methoxy-2,2-dimethyl-N-(pyridin-3-ylmethyl)-1,2,3,4-tetrahydropyrimido[4′,5′:4,5]furo[2,3-c]isoquinolin-8-amine (140 mg, 0.36 mmol, see Example 1) is dissolved in bromhydric acid (5 ml, 48% wt. in water) and the mixture is heated at 10° C. for 3 h. Once at room temperature, the reaction mixture is neutralised with NaOH 6N, precipitating a solid, which is filtered and dried. 0.13 g of the final compound is obtained. Yield=96%.

LRMS: m/z 376 (M+1)⁺.

### Example 3

### 2,2-Dimethyl-5-morpholin-4-yl-N-(pyridin-3-ylmethyl)-1,2,3,4-tetrahydropyrimido[4′,5′:4,5]furo[2,3-c]isoquinolin-8-amine

2,2-Dimethyl-8-[(pyridin-3-ylmethyl)amino]-1,2,3,4-tetrahydropyrimido[4′,5′:4,5]furo[2,3-c]isoquinolin-5-ol (25 mg, 0.07 mmol, see Example 2) dissolved in DMF (2 ml) is dropwise added to a suspension of sodium hydride (60% dispersion in mineral oil, 2.7 mg, 6.7 mmol) in DMF (2 ml). Then, N-phenylbistrifluorometansulfonamide (2.4 mg, 0.07 mmol) is added and the reaction mixture is stirred during one hour (it turns red). Morpholine (0.01 ml, 0.13 mmol) is then added and the reaction is stirred overnight. The solvent is evaporated under reduced pressure and the residue is redissolved in chloroform and water. The organic phase is separated and the aqueous phase is extracted twice with chloroform. The organic phase is washed with water and brine, dried over magnesium sulfate, filtered and evaporated. The residue is purified by flash chromatography, eluting with CH₂Cl₂/MeOH 98:2. 20 mg of the final compound are obtained. Yield=67%.

¹H NMR (400 MHz, CHLOROFORM-D) δ ppm 1.1 (s, 6H) 1.6 (t, J=6.2 Hz, 2H) 2.8 (t, J=6.4 Hz, 2H) 3.2 (s, 2H) 3.3 (m, 4H) 3.9 (m, 4H) 4.9 (d, J=6.2 Hz, 2H) 5.5 (t, J=6.0 Hz, 1H) 7.3 (dd, J=7.9, 5.0 Hz, 1H) 7.7 (m, 1H) 8.6 (dd, J=5.0, 1.7 Hz, 1H) 8.7 (s, 1H) 8.7 (d, J=2.1 Hz, 1H).

### Example 4

### 2,2-Dimethyl-N-(pyridin-3-ylmethyl)-5-pyrrolidin-1-yl-1,2,3,4-tetrahydropyrimido[4′,5′:4,5]furo[2,3-c]isoquinolin-8-amine

Obtained (68%) from the title compound of Example 2 and pyrrolidine following the experimental procedure described in Example 3.

¹H NMR (400 MHz, CHLOROFORM-D) δ ppm 1.1 (s, 6H) 1.6 (t, J=6.5 Hz, 2H) 2.0 (m, 4H) 2.8 (t, J=6.5 Hz, 2H) 3.2 (s, 2H) 3.7 (m, 4H) 4.9 (d, J=5.9 Hz, 2H) 5.4 (t, J=5.9 Hz, 1H) 7.3 (m, 1H) 7.7 (dd, J=7.8, 2.0 Hz, 1H) 8.5 (d, J=3.5 Hz, 1H) 8.6 (s, 1H) 8.7 (s, 1H).

### Example 5

### N⁵,N⁵,2,2-Tetramethyl-N⁸-(pyridin-3-ylmethyl)-1,2,3,4-tetrahydropyrimido[4′,5′:4,5]furo[2,3-c]isoquinoline-5,8-diamine

Obtained (41%) from the title compound of Example 2 and dimethylamine following the experimental procedure described in Example 3.

LRMS: m/z 403 (M+1)⁺.

### Example 6

### N⁵-Ethyl-N⁵,2,2-trimethyl-N⁸-(pyridin-3-ylmethyl)-1,2,3,4-tetrahydropyrimido[4′,5′:4,5]furo[2,3-c]isoquinoline-5,8-diamine

Obtained (34%) from the title compound of Example 2 and ethylmethylamine following the experimental procedure described in Example 3.

LRMS: m/z 417 (M+1)⁺.

### Example 7

### 2,2-Dimethyl-5-morpholin-4-yl-N-(2-morpholin-4-ylethyl)-1,4-dihydro-2H-pyrano[4″,3″:4′,5′]pyrido[3′,2′:4,5]furo[3,2-d]pyrimidin-8-amine

8-Chloro-2,2-dimethyl-5-morpholin-4-yl-1,4-dihydro-2H-pyrano[4″,3″:4′,5′]pyrido[3′,2′:4,5]furo[3,2-d]pyrimidine (70 mg, 0.2 mmol, see Preparation 18) is suspended in ethanol (5 ml) and 2-morpholine-4-ylethylamine (123 μl, 0.9 mmol) is added. The reaction mixture is heated at 85° C. for 48 h. The solvent is evaporated under reduced pressure and the residue is passed through a silica-gel column eluting dichloromethane first and then successively with the mixtures CH₂Cl₂/MeOH 99:1 and 98:2. 50 mg of the final compound are obtained. Yield=57%.

¹H NMR (400 MHz, DMSO-D6) δ ppm 1.3 (s, 6H) 2.4 (m, 4H) 2.5 (d, J=2.0 Hz, 3H) 2.6 (t, J=6.8 Hz, 2H) 3.2 (m, 4H) 3.3 (s, 2H) 3.6 (m, 4H) 3.8 (m, 4H) 4.7 (s, 2H) 8.4 (s, 1H).

### Example 8

### 2,2-Dimethyl-5-morpholin-4-yl-N-(pyridin-3-ylmethyl)-1,4-dihydro-2H-pyrano[4″,3″:4′,5′]pyrido[3′,2′:4,5]furo[3,2-d]pyrimidin-8-amine

Obtained (22%) from the title compound of Preparation 18 and pyridine-3-ylmethylamine following the experimental procedure described in Example 7.

¹H NMR (400 MHz, DMSO-D6) δ ppm 1.3 (s, 6H) 3.2 (m, 4H) 3.3 (s, 2H) 3.8 (m, 4H) 4.7 (s, 2H) 4.8 (d, J=5.9 Hz, 2H) 7.3 (dd, J=7.8, 4.7 Hz, 1H) 7.8 (m, 1H) 8.5 (m, 1H) 8.6 (m, 2H).

### Example 9

### N-(2,3-Dimethoxybenzyl)-2,2-dimethyl-5-morpholin-4-yl-1,4-dihydro-2H-pyrano[4″,3″:4′,5′]pyrido[3′,2′:4,5]furo[3,2-d]pyrimidin-8-amine

Obtained (67%) from the title compound of Preparation 18 and 2,3-dimethoxybenzylamine following the experimental procedure described in Example 7.

¹H NMR (400 MHz, DMSO-D6) δ ppm 1.3 (s, 6H) 3.2 (m, 4H) 3.3 (s, 2H) 3.8 (m, 4H) 3.8 (s, 6H) 4.7 (s, 2H) 4.8 (d, J=5.9 Hz, 2H) 6.9 (dd, J=7.4, 2.0 Hz, 1H) 6.9 (m, 2H) 8.4 (s, 1H) 8.4 (bs, 1H).

### Example 10

### 2-[(2,2-Dimethyl-5-morpholin-4-yl-1,4-dihydro-2H-pyrano[4″,3″:4′,5′]pyrido[3′,2′:4,5]furo[3,2-d]pyrimidin-8-yl)(2-morpholin-4-ylethyl)amino]ethanol

Obtained (57%) from the title compound of Preparation 18 and 2-(2-morpholin-4-ylethylamino)ethanol following the experimental procedure described in Example 7.

¹H NMR (400 MHz, DMSO-D6) δ ppm 1.3 (s, 6H) 2.6 (m, 2H) 3.2 (s, 4H) 3.3 (s, 2H) 3.5 (s, 4H) 3.7 (m, 9H) 4.0 (s, 4H) 4.7 (s, 2H) 4.9 (m, 2H) 8.4 (s, 1H).

### Example 11

N⁵,N⁵,2,2-Tetramethyl-N⁸-(2-morpholin-4-ylethyl)-1,4-dihydro-2H-pyrano[4″,3″:4′,5′]pyrido[3′,2′:4,5]furo[3,2-d]pyrimidine-5,8-diamine

Obtained (58%) from the title compound of Preparation 25 and 2-morpholin-4-ylethylamine following the experimental procedure described in Example 7.

¹H NMR (400 MHz, DMSO-D6) δ ppm 1.3 (s, 6H) 2.4 (s, 4H) 2.6 (t, J=6.8 Hz, 2H) 2.9 (s, 6H) 3.2 (s, 2H) 3.6 (m, 6H) 4.7 (s, 2H) 7.8 (m, 1H) 8.4 (s, 1H).

### Example 12

### N⁵,N⁵,2,2-Tetramethyl-N⁸-(pyridin-3-ylmethyl)-1,4-dihydro-2H-pyrano[4″,3″:4′,5′]pyrido[3′,2′:4,5]furo[3,2-d]pyrimidine-5,8-diamine

Obtained (57%) from the title compound of Preparation 25 and pyridin-3-ylmethylamine following the experimental procedure described in Example 7.

¹H NMR (400 MHz, DMSO-D6) δ ppm 1.3 (s, 6H) 2.9 (s, 6H) 3.2 (s, 2H) 4.7 (s, 2H) 4.7 (d, J=6.3 Hz, 2H) 7.3 (dd, J=7.8, 4.3 Hz, 1H) 7.7 (d, J=7.8 Hz, 1H) 8.4 (s, 1H) 8.4 (dd, J=4.7, 1.6 Hz, 1H) 8.5 (t, J=6.3 Hz, 1H) 8.6 (d, J=2.0 Hz, 1H).

### Example 13

### N⁸-(2,3-Dimethoxybenzyl)-N⁵,N⁵,2,2-tetramethyl-1,4-dihydro-2H-pyrano[4″,3″:4′,5′]pyrido[3′,2′:4,5]furo[3,2-d]pyrimidine-5,8-diamine

Obtained (70%) from the title compound of Preparation 25 and 2,3-dimethoxybenzylamine following the experimental procedure described in Example 7.

¹H NMR (400 MHz, DMSO-D6) δ ppm 1.3 (s, 6H) 2.9 (s, 6H) 3.2 (s, 2H) 3.8 (s, 6H) 4.7 (s, 2H) 4.7 (d, J=6.3 Hz, 2H) 6.8 (d, J=7.4 Hz, 1H) 6.9 (m, 2H) 8.3 (m, 1H) 8.4 (s, 1H).

### Example 14

### 2,2-Dimethyl-5-morpholin-4-yl-N-(2-morpholin-4-ylethyl)-N-(pyridin-3-ylmethyl)-1,4-dihydro-2H-pyrano[4″,3″:4′,5′]pyrido[3′,2′:4,5]furo[3,2-d]pyrimidin-8-amine

Obtained (32%) from the title compound of Preparation 18 and (2-morpholin-4-ylethyl)pyridin-3-ylmethylamine following the experimental procedure described in Example 7.

¹H NMR (300 MHz, DMSO-D6, fumarate) δ ppm 1.3 (s, 6H) 2.4 (s, 2H) 2.6 (t, J=6.3 Hz, 2H) 3.2 (m, 4H) 3.5 (m, 4H) 3.7 (m, 4H) 3.9 (s, 2H) 4.5 (s, 4H) 4.7 (s, 2H) 5.1 (s, 2H) 6.6 (s, 2H) 7.4 (m, 1H) 7.7 (d, J=7.7 Hz, 1H) 8.5 (m, 2H) 8.5 (d, J=1.4 Hz, 1H) 8.6 (d, J=1.9 Hz, 1H).

### Example 15

### N5 N⁵,2,2-tetramethyl-N⁸-(2-morpholin-4-ylethyl)-N⁸-(pyridin-3-ylmethyl)-1,4-dihydro-2H-pyrano[4″,3″:4′,5′]pyrido[3′,2′:4,5]furo[3,2-d]pyrimidine-5,8-diamine

Obtained (39%) from the title compound of Preparation 25 and (2-morpholin-4-ylethyl)pyridin-3-ylmethylamine following the experimental procedure described in Example 7.

¹H NMR (300 MHz, DMSO-D6) δ ppm 1.3 (s, 6H) 2.4 (s, 4H) 2.6 (t, J=6.5 Hz, 2H) 2.9 (s, 6H) 3.5 (m, 4H) 3.9 (t, J=6.2 Hz, 2H) 4.5 (s, 2H) 4.7 (s, 2H) 5.1 (s, 2H) 6.6 (s, 2H) 7.4 (m, 1H) 7.7 (d, J=7.7 Hz, 1H) 8.5 (m, 2H) 8.6 (d, J=1.6 Hz, 1H).

### Example 16

### N⁸-(3,4-dimethoxybenzyl)-N⁵,N⁵,2,2-tetramethyl-1,4-dihydro-2H-pyrano[4″,3″:4′,5′]pyrido[3′,2′:4,5]furo[3,2-d]pyrimidine-5,8-diamine

Obtained (60%) from the title compound of Preparation 25 and 3,4-dimethoxybenzylamine following the experimental procedure described in Example 7.

¹H NMR (300 MHz, CHLOROFORM-D) δ ppm 1.4 (s, 6H) 3.0 (s, 6H) 3.4 (s, 2H) 3.9 (m, J=2.2 Hz, 6H) 4.8 (m, 4H) 5.4 (m, 1H) 6.8 (m, 1H) 6.9 (m, 2H) 8.6 (s, 1H).

### Example 17

### 5-Methoxy-2,2-dimethyl-N-(2-morpholin-4-ylethyl)-1,2,3,4-tetrahydropyrimido[4′,5′:4,5]furo[2,3-c]isoquinolin-8-amine

Obtained (84%) from the title compound of Preparation 7 and 2-morpholin-4-ylethylamine following the experimental procedure described in Example 1.

¹H NMR (300 MHz, CHLOROFORM-D) δ ppm 1.1 (s, 6H) 1.7 (t, J=6.5 Hz, 2H) 2.5 (m, 4H) 2.7 (m, 4H) 3.2 (s, 2H) 3.8 (m, 6H) 4.1 (s, 3H) 5.9 (s, 1H) 8.6 (s, 1H).

### Example 18

### 1-{3-[(2,2-Dimethyl-5-morpholin-4-yl-1,2,3,4-tetrahydropyrimido[4′,5′:4,5]furo[2,3-c]isoquinolin-8-yl)amino]propyl}pyrrolidin-2-one

8-Chloro-2,2-dimethyl-5-morpholin-4-yl-1,2,3,4-tetrahydropyrimido[4′,5′:4,5]furo[2,3-c]isoquinoline (90 mg, 0.2 mmol, see Preparation 32) is suspended in ethanol (5 ml) and 1-(3-aminopropyl)-pyrrolidin-2-one (169 μl, 1.2 mmol) is added. The reaction mixture is heated at 85° C. for 48 h. The solvent is evaporated under reduced pressure and the residue is passed through a silica-gel column eluting first with dichloromethane and then successively with the mixtures CH₂Cl₂/MeOH 99:1 and 98:2. 40 mg of the final compound are obtained. Yield=34%.

¹H NMR (300 MHz, CHLOROFORM-D) δ ppm 1.1 (s, 6H) 1.6 (m, 2H) 1.9 (t, J=6.5 Hz, 2H) 2.1 (m, 2H) 2.5 (t, J=8.1 Hz, 2H) 2.7 (m, 2H) 3.2 (s, 2H) 3.3 (m, 4H) 3.4 (t, J=7.0 Hz, 4H) 3.7 (m, 2H) 3.9 (m, 4H) 6.2 (s, 1H) 8.6 (s, 1H).

### Example 19

### 2,2-Dimethyl-8-[(2-morpholin-4-ylethyl)amino]-1,2,3,4-tetrahydropyrimido[4′,5′:4,5]furo[2,3-c]isoquinolin-5-ol

5-Methoxy-2,2-dimethyl-N-(2-morpholin-4-ylethyl)-1,2,3,4-tetrahydropyrimido[4′,5′:4,5]furo[2,3-c]isoquinolin-8-amine (1.1 g, 2.7 mmol, see Example 17) is dissolved in bromhydric acid (48% wt. in water) and the mixture is heated at 100° C. for 3 h. Once at room temperature, the reaction mixture is neutralised with NaOH 5N, precipitating a solid, which is filtered and dried. 0.98 g of the final compound is obtained. Yield=93%.

¹H NMR (300 MHz, DMSO-D6) δ ppm 1.0 (s, 6H) 1.6 (t, J=6.0 Hz, 2H) 2.5 (s, 4H) 2.6 (t, J=6.5 Hz, 2H) 3.1 (s, 4H) 3.6 (m, 6H) 8.0 (s, 1H) 8.5 (s, 1H) 10.1 (s, 1H).

### Example 20

### 2-[(2,2-Dimethyl-5-morpholin-4-yl-1,2,3,4-tetrahydropyrimido[4′,5′:4,5]furo[2,3-c]isoquinolin-8-yl)(2-morpholin-4-ylethyl)amino]ethanol

Obtained (16%) from the title compound of Preparation 32 and 2-(2-morpholin-4-ylethylamino)ethanol following the experimental procedure described in Example 1.

LRMS: m/z 511 (M+1)⁺.

### Example 21

### 1-{3-[(5-Methoxy-2,2-dimethyl-1,2,3,4-tetrahydropyrimido[4′,5′:4,5]furo[2,3-c]isoquinolin-8-yl)amino]propyl}pyrrolidin-2-one

Obtained (71%) from the title compound of Preparation 7 and 1-(3-aminopropyl)-pyrrolidin-2-one following the experimental procedure described in Example 1.

¹H NMR (300 MHz, CHLOROFORM-D) δ ppm 1.1 (s, 6H) 1.7 (t, J=6.7 Hz, 2H) 1.9 (m, 2H) 2.1 (m, 2H) 2.5 (t, J=8.1 Hz, 2H) 2.7 (t, J=6.6 Hz, 2H) 3.2 (s, 2H) 3.4 (m, 4H) 3.7 (m, J=6.3, 6.3, 6.3 Hz, 2H) 4.1 (s, 3H) 6.2 (s, 1H) 8.6 (s, 1H).

### Example 22

### N-(2,3-Dimethoxybenzyl)-2,2-dimethyl-5-morpholin-4-yl-1,2,3,4-tetrahydropyrimido[4′,5′:4,5]furo[2,3-c]isoquinolin-8-amine

Obtained (85%) from the title compound of Preparation 32 and 2,3-dimethoxybenzylamine following the experimental procedure described in Example 18.

¹H NMR (300 MHz, CHLOROFORM-D) δ ppm 1.1 (s, 6H) 1.6 (t, J=6.2 Hz, 2H) 2.7 (t, J=6.2 Hz, 2H) 3.2 (s, 2H) 3.3 (m, 4H) 3.9 (m, 7H) 3.9 (m, J=5.2 Hz, 3H) 4.9 (m, J=6.5, 6.5 Hz, 2H) 5.7 (m, 1H) 6.9 (dd, J=7.1, 2.5 Hz, 1H) 7.0 (m, 2H) 8.7 (s, 1H).

### Example 23

### 2,2-Dimethyl-5-morpholin-4-yl-N-(2-morpholin-4-ylethyl)-1,2,3,4-tetrahydropyrimido[4′,5′:4,5]furo[2,3-c]isoquinolin-8-amine

Obtained (35%) from the title compound of Example 19 and morpholine following the experimental procedure described in Example 3.

¹H NMR (300 MHz, CHLOROFORM-D) δ ppm 1.1 (s, 6H) 1.6 (m, 2H) 2.5 (s, 4H) 2.7 (m, 2H) 2.8 (t, J=6.2 Hz, 2H) 3.2 (s, 2H) 3.3 (m, 4H) 3.8 (m, 6H) 3.9 (m, 4H) 5.9 (t, J=4.8 Hz, 1H) 8.6 (s, 1H).

### Example 24

### 2,2-Dimethyl-N-(2-morpholin-4-ylethyl)-5-pyrrolidin-1-yl-1,2,3,4-tetrahydropyrimido[4′,5′:4,5]furo[2,3-c]isoquinolin-8-amine

Obtained (31%) from the title compound of Example 19 and pyrrolidine following the experimental procedure described in Example 3.

¹H NMR (300 MHz, CHLOROFORM-D) δ ppm 1.1 (s, 6H) 1.6 (t, J=6.3 Hz, 2H) 2.0 (m, 4H) 2.5 (s, 4H) 2.7 (m, 2H) 2.8 (t, J=6.3 Hz, 2H) 3.2 (s, 2H) 3.7 (m, 6H) 3.8 (m, 4H) 5.8 (t, J=4.8 Hz, 1H) 8.6 (s, 1H).

### Example 25

### N⁵,N⁵,2,2-Tetramethyl-N⁸-(2-morpholin-4-ylethyl)-1,2,3,4-tetrahydropyrimido [4′,5′:4,5]furo[2,3-c]isoquinoline-5,8-diamine

Obtained (43%) from the title compound of Example 19 and dimethylamine following the experimental procedure described in Example 3.

¹H NMR (300 MHz, CHLOROFORM-D) δ ppm 1.1 (s, 6H) 1.6 (m, 2H) 2.5 (s, 4H) 2.7 (m, 2H) 2.8 (t, J=6.3 Hz, 2H) 3.0 (s, 6H) 3.2 (s, 2H) 3.8 (m, 6H) 5.9 (t, J=4.5 Hz, 1H) 8.6 (s, 1H).

### Example 26

### 2,2-Dimethyl-8-{[3-(2-oxopyrrolidin-1-yl)propyl]amino}-2,3,4,6-tetrahydropyrimido[4′,5′:4,5]furo[2,3-c]isoquinolin-5(1H)-one

1-{3-[(5-Methoxy-2,2-dimethyl-1,2,3,4-tetrahydropyrimido[4′,5′:4,5]furo[2,3-c]isoquinolin-8-yl)amino]propyl}pyrrolidin-2-one (0.9 g, 2.1 mmol, see Example 21) is dissolved in bromhydric acid (48% wt. in water) and the mixture is heated at 100° C. for 3 h. Once at room temperature, the reaction mixture is neutralised with NaOH 8N, precipitating a solid. Dimethylformamide is added to this aqueous solution until the solid is completely dissolved. This aqueous solution is extracted four times with chloroform and the organic phase is washed with brine, dried over magnesium sulfate and filtered. The solvent is evaporated under reduced pressure and the residue is rinsed with ethyl ether, filtering the resulting solid. 880 g of the final compound are obtained. Yield=100%.

LRMS: m/z 410 (M+1)⁺.

### Example 27

### 2-({2,2-Dimethyl-8-[(2-morpholin-4-ylethyl)amino]-1,2,3,4-tetrahydropyrimido[4′,5′:4,5]furo[2,3-c]isoquinolin-5-yl}amino)ethanol

Obtained (18%) from the title compound of Example 19 and 2-aminoethanol following the experimental procedure described in Example 3.

¹H NMR (300 MHz, CHLOROFORM-D) δ ppm 1.1 (s, 6H) 1.7 (t, J=6.6 Hz, 2H) 2.5 (t, J=6.5 Hz, 2H) 2.6 (m, 4H) 2.7 (s, 2H) 3.1 (s, 2H) 3.8 (m, 8H) 3.9 (m, 2H) 5.1 (t, J=5.8 Hz, 1H) 5.8 (s, 1H) 8.6 (s, 1H).

### Example 28

### 2,2-Dimethyl-N,N′-bis(2-morpholin-4-ylethyl)-1,2,3,4-tetrahydropyrimido[4′,5′:4,5]furo[2,3-c]isoquinoline-5,8-diamine

Obtained (35%) from the title compound of Example 19 and 2-morpholin-4-ylethylamine following the experimental procedure described in Example 3.

¹H NMR (300 MHz, CHLOROFORM-D) δ ppm 1.1 (s, 6H) 1.7 (t, J=6.6 Hz, 2H) 2.4 (t, J=6.6 Hz, 2H) 2.5 (m, 8H) 2.7 (m, 2H) 2.7 (m, 2H) 3.1 (s, 2H) 3.6 (m, 2H) 3.7 (m, 2H) 3.8 (m, 8H) 5.6 (s, 1H) 5.8 (t, J=4.5 Hz, 1H) 8.6 (s, 1H).

### Example 29

### 1-(3-{[5-(Dimethylamino)-2,2-dimethyl-1,2,3,4-tetrahydropyrimido[4′,5′:4,5]furo[2,3-c]isoquinolin-8-yl]amino}propyl)pyrrolidin-2-one

Obtained (54%) from the title compound of Example 26 and dimethylamine following the experimental procedure described in Example 3.

¹H NMR (300 MHz, CHLOROFORM-D) δ ppm 1.1 (s, 6H) 1.6 (t, J=6.3 Hz, 2H) 1.9 (m, 2H) 2.1 (m, 2H) 2.4 (t, J=8.1 Hz, 2H) 2.7 (t, J=6.2 Hz, 2H) 3.0 (s, 6H) 3.2 (s, 2H) 3.4 (m, 4H) 3.7 (q, J=6.3 Hz, 2H) 6.0 (t, J=6.2 Hz, 1H) 8.6 (s, 1H).

### Example 30

### 1-{3-[(2,2-Dimethyl-5-pyrrolidin-1-yl-1,2,3,4-tetrahydropyrimido[4′,5′:4,5]furo[2,3-c]isoquinolin-8-yl)amino]propyl}pyrrolidin-2-one

Obtained (45%) from the title compound of Example 26 and pyrrolidine following the experimental procedure described in Example 3.

¹H NMR (300 MHz, CHLOROFORM-D) δ ppm 1.1 (s, 6H) 1.6 (t, J=6.3 Hz, 2H) 1.9 (t, 2H) 2.0 (m, 4H) 2.1 (m, 2H) 2.4 (t, J=8.1 Hz, 2H) 2.8 (t, J=6.3 Hz, 2H) 3.2 (s, 2H) 3.4 (m, 4H) 3.7 (m, 6H) 5.8 (t, J=6.3 Hz, 1H) 8.6 (s, 1H).

### Example 31

### N⁵-Ethyl-2,2-dimethyl-N⁸-(2-morpholin-4-ylethyl)-1,2,3,4-tetrahydropyrimido[4′,5′:4,5]furo[2,3-c]isoquinoline-5,8-diamine

Obtained (36%) from the title compound of Example 19 and ethylamine following the experimental procedure described in Example 3.

¹H NMR (300 MHz, CHLOROFORM-D) δ ppm 1.1 (s, 6H) 1.4 (t, J=7.1 Hz, 3H) 1.7 (t, J=6.6 Hz, 2H) 2.4 (t, J=6.3 Hz, 2H) 2.5 (s, 4H) 2.7 (t, J=5.5 Hz, 2H) 3.1 (s, 2H) 3.6 (m, 2H) 3.8 (m, 6H) 4.6 (t, J=5.2 Hz, 1H) 5.8 (t, J=4.0 Hz, 1H) 8.6 (s, 1H).

### Example 32

### N⁵-Ethyl-N⁵,2,2-trimethyl-N⁸-(2-morpholin-4-ylethyl)-1,2,3,4-tetrahydropyrimido [4′,5′:4,5]furo[2,3-c]isoquinoline-5,8-diamine

Obtained (41%) from the title compound of Example 19 and ethyl(methyl)amine following the experimental procedure described in Example 3.

¹H NMR (300 MHz, CHLOROFORM-D) δ ppm 1.1 (s, 6H) 1.2 (t, J=7.1 Hz, 3H) 1.6 (t, 2H) 2.5 (m, 4H) 2.7 (m, 4H) 3.0 (s, 3H) 3.2 (s, 2H) 3.3 (q, J=6.9 Hz, 2H) 3.7 (m, 6H) 5.9 (t, J=4.8 Hz, 1H) 8.6 (s, 1H).

### Example 33

### N⁵-Isopropyl-2,2-dimethyl-N⁸-(2-morpholin-4-ylethyl)-1,2,3,4-tetrahydropyrimido[4′,5′:4,5]furo[2,3-c]isoquinoline-5,8-diamine

Obtained (18%) from the title compound of Example 19 and diisopropylamine following the experimental procedure described in Example 3.

¹H NMR (300 MHz, CHLOROFORM-D) δ ppm 1.1 (s, 6H) 1.3 (d, J=6.0 Hz, 6H) 1.7 (t, J=6.7 Hz, 2H) 2.4 (t, J=6.6 Hz, 2H) 2.5 (m, 4H) 2.7 (m, 2H) 3.1 (s, 2H) 3.7 (m, 7H) 4.5 (m, 1H) 5.8 (t, J=4.8 Hz, 1H) 8.6 (s, 1H).

### Example 34

### 1-[3-({5-[(2-Hydroxyethyl)amino]-2,2-dimethyl-1,2,3,4-tetrahydropyrimido[4′,5′:4,5]furo[2,3-c]isoquinolin-8-yl}amino)propyl]pyrrolidin-2-one

Obtained (23%) from the title compound of Example 26 and (2-hydroxyethyl)amine following the experimental procedure described in Example 3.

¹H NMR (300 MHz, CHLOROFORM-D) δ ppm 1.1 (s, 6H) 1.7 (m, 2H) 1.9 (m, 2H) 2.1 (m, 2H) 2.4 (m, 4H) 3.1 (s, 2H) 3.4 (m, 4H) 3.7 (m, 2H) 3.8 (m, 2H) 3.9 (m, 2H) 5.1 (t, J=4.8 Hz, 1H) 6.0 (t, J=5.9 Hz, 1H) 8.5 (s, 1H).

### Example 35

### 1-(3-{[5-(Ethylamino)-2,2-dimethyl-1,2,3,4-tetrahydropyrimido[4′,5′:4,5]furo[2,3-c]isoquinolin-8-yl]amino}propyl)pyrrolidin-2-one

Obtained (32%) from the title compound of Example 26 and ethylamine following the experimental procedure described in Example 3.

¹H NMR (300 MHz, CHLOROFORM-D) δ ppm 1.1 (s, 6H) 1.3 (t, J=7.3 Hz, 3H) 1.7 (t, J=6.5 Hz, 2H) 1.9 (m, 2H) 2.1 (m, 2H) 2.4 (m, 4H) 3.1 (s, 2H) 3.4 (m, 4H) 3.6 (m, 4H) 4.6 (t, J=4.3 Hz, 1H) 5.8 (s, 1H) 8.6 (s, 1H).

### Example 36

### 1-[3-({5-[Ethyl(methyl)amino]-2,2-dimethyl-1,2,3,4-tetrahydropyrimido[4′,5′:4,5]furo[2,3-c]isoquinolin-8-yl}amino)propyl]pyrrolidin-2-one

Obtained (66%) from the title compound of Example 26 and ethyl(methyl)amine following the experimental procedure described in Example 3.

¹H NMR (300 MHz, CHLOROFORM-D) δ ppm 1.1 (s, 6H) 1.2 (t, J=7.1 Hz, 3H) 1.6 (t, J=6.5 Hz, 2H) 1.9 (m, 2H) 2.1 (m, 2H) 2.4 (t, J=8.1 Hz, 2H) 2.7 (t, J=6.3 Hz, 2H) 3.0 (s, 3H) 3.2 (s, 2H) 3.3 (q, J=7.1 Hz, 2H) 3.4 (m, 4H) 3.7 (q, J=6.4 Hz, 2H) 6.0 (s, 1H) 8.6 (s, 1H).

### Example 37

### 2-[[5-(Dimethylamino)-2,2-dimethyl-1,4-dihydro-2H-pyrano[4″,3″:4′,5′]pyrido[3′,2′:4,5]furo[3,2-d]pyrimidin-8-yl](2-morpholin-4-ylethyl)amino]ethanol

Obtained (83%) from the title compound of Preparation 25 and 2-(2-morpholin-4-ylethylamino)ethanol following the experimental procedure described in Example 7.

¹H NMR (300 MHz, CHLOROFORM-D) δ ppm 1.4 (s, 6H) 2.6 (s, 4H) 2.9 (s, 2H) 3.0 (s, 6H) 3.4 (s, 2H) 3.8 (m, 4H) 4.0 (m, 6H) 4.8 (s, 2H) 8.5 (s, 1H).

### Example 38

### 1-(3-{[5-(Isopropylamino)-2,2-dimethyl-1,2,3,4-tetrahydropyrimido[4′,5′:4,5]furo[2,3-c]isoquinolin-8-yl]amino}propyl)pyrrolidin-2-one

Obtained (56%) from the title compound of Example 26 and dimethylamine following the experimental procedure described in Example 3.

¹H NMR (300 MHz, CHLOROFORM-D) δ ppm 1.1 (s, 6H) 1.3 (s, 6H) 1.7 (m, 2H) 1.9 (m, 2H) 2.1 (m, 2H) 2.4 (m, 4H) 3.1 (s, 2H) 3.4 (m, 4H) 3.7 (m, 2H) 4.4 (m, 2H) 5.8 (s, 1H) 8.6 (s, 1H).

### Example 39

### 2-[(5-Methoxy-2,2-dimethyl-1,2,3,4-tetrahydropyrimido[4′,5′:4,5]furo[2,3-c]isoquinolin-8-yl)(2-morpholin-4-ylethyl)amino]ethanol

Obtained (69%) from the title compound of Preparation 7 and 2-(2-morpholin-4-ylethylamino)ethanol following the experimental procedure described in Example 7.

¹H NMR (300 MHz, CHLOROFORM-D) δ ppm 1.1 (s, 6H) 1.7 (t, J=6.7 Hz, 2H) 2.6 (s, 4H) 2.7 (t, J=6.5 Hz, 2H) 2.9 (s, 2H) 3.2 (s, 2H) 3.7 (m, 4H) 4.0 (m, 9H) 8.6 (s, 1H).

### Example 40

### 8-[(2-Hydroxyethyl)(2-morpholin-4-ylethyl)amino]-2,2-dimethyl-1,2,3,4-tetrahydropyrimido[4′,5′:4,5]furo[2,3-c]isoquinolin-5-ol

Obtained (46%) from the title compound of Example 39 following the experimental procedure described in Example 21.

¹H NMR (300 MHz, DMSO-D6) δ ppm 1.0 (s, 6H) 1.6 (t, J=6.3 Hz, 2H) 2.6 (m, 4H) 3.1 (s, 2H) 3.3 (m, 2H) 3.7 (m, 6H) 3.9 (s, 4H) 4.1 (s, 4H) 8.4 (s, 1H).

### Example 41

### N⁵-Cyclopropyl-2,2-dimethyl-N″-(2-morpholin-4-ylethyl)-1,2,3,4-tetrahydropyrimido[4′,5′:4,5]furo[2,3-c]isoquinoline-5,8-diamine

Obtained (44%) from the title compound of Example 19 and cyclopropylamine following the experimental procedure described in Example 3.

¹H NMR (300 MHz, CHLOROFORM-D) δ ppm 0.6 (m, 2H) 1.0 (m, 2H) 1.1 (s, 6H) 1.7 (t, J=6.5 Hz, 2H) 2.4 (t, J=6.5 Hz, 2H) 2.5 (s, 4H) 2.7 (m, 2H) 2.9 (m, 1H) 3.1 (s, 2H) 3.7 (m, 2H) 3.8 (m, 4H) 4.9 (d, J=1.1 Hz, 1H) 5.8 (t, J=5.1 Hz, 1H) 8.6 (s, 1H).

### Example 42

### N⁵-Cyclopentyl-2,2-dimethyl-N⁸-(2-morpholin-4-ylethyl)-1,2,3,4-tetrahydropyrimido[4′,5′:4,5]furo[2,3-c]isoquinoline-5,8-diamine

Obtained (23%) from the title compound of Example 19 and cyclopentylamine following the experimental procedure described in Example 3.

¹H NMR (300 MHz, CHLOROFORM-D) δ ppm 1.1 (s, 6H) 1.5 (m, 2H) 1.7 (m, 6H) 2.2 (m, 2H) 2.4 (t, J=6.5 Hz, 2H) 2.5 (m, 4H) 2.7 (m, 2H) 3.1 (s, 2H) 3.7 (m, 2H) 3.8 (m, 4H) 4.5 (q, J=6.7 Hz, 1H) 4.6 (m, 1H) 5.8 (t, J=4.7 Hz, 1H) 8.6 (s, 1H).

### Example 43

### N⁵-Ethyl-2,2-dimethyl-N⁸-(pyridin-3-ylmethyl)-1,2,3,4-tetrahydropyrimido[4′,5′:4,5]furo[2,3-c]isoquinoline-5,8-diamine

Obtained (33%) from the title compound of Example 2 and ethylamine following the experimental procedure described in Example 3.

¹H NMR (300 MHz, CHLOROFORM-D) δ ppm 1.1 (s, 6H) 1.3 (t, J=7.3 Hz, 3H) 1.7 (t, J=6.5 Hz, 2H) 2.4 (t, J=6.2 Hz, 2H) 3.1 (s, 2H) 3.6 (m, 2H) 4.7 (t, J=4.5 Hz, 1H) 4.9 (d, J=6.0 Hz, 2H) 5.5 (t, J=5.6 Hz, 1H) 7.3 (m, 1H) 7.7 (d, J=8.0 Hz, 1H) 8.6 (d, J=4.7 Hz, 1H) 8.6 (s, 1H) 8.7 (s, 1H).

### Example 44

### N⁵-Isopropyl-2,2-dimethyl-N⁸-(pyridin-3-ylmethyl)-1,2,3,4-tetrahydropyrimido[4′,5′:4,5]furo[2,3-c]isoquinoline-5,8-diamine

Obtained (36%) from the title compound of Example 2 and isopropylamine following the experimental procedure described in Example 3.

¹H NMR (300 MHz, CHLOROFORM-D) δ ppm 1.1 (s, 6H) 1.3 (d, J=6.6 Hz, 6H) 1.7 (t, J=6.6 Hz, 2H) 2.4 (t, J=6.5 Hz, 2H) 3.1 (s, 2H) 4.4 (m, 1H) 4.5 (m, 1H) 4.9 (d, J=6.0 Hz, 2H) 5.4 (t, J=5.9 Hz, 1H) 7.3 (dd, J=4.5, 3.4 Hz, 1H) 7.7 (d, J=7.7 Hz, 1H) 8.6 (d, J=3.8 Hz, 1H) 8.6 (s, 1H) 8.7 (s, 1H).

### Example 45

### 2-({2,2-Dimethyl-8-[(pyridin-3-ylmethyl)amino]-1,2,3,4-tetrahydropyrimido[4′,5′:4,5]furo[2,3-c]isoquinolin-5-yl}amino)ethanol

Obtained (44%) from the title compound of Example 2 and 2-aminoethanol following the experimental procedure described in Example 3.

¹H NMR (300 MHz, CHLOROFORM-D) δ ppm 1.1 (s, 6H) 1.7 (s, 2H) 2.5 (s, 2H) 3.1 (s, 2H) 3.8 (s, 2H) 3.9 (s, 2H) 4.9 (d, J=5.8 Hz, 2H) 5.2 (s, 1H) 5.5 (s, 1H) 7.3 (d, J=5.8 Hz, 1H) 7.8 (d, J=6.0 Hz, 1H) 8.6 (d, J=4.1 Hz, 1H) 8.6 (s, 1H) 8.7 (s, 1H).

### Example 46

### N⁵-Cyclobutyl-2,2-dimethyl-N⁸-(2-morpholin-4-ylethyl)-1,2,3,4-tetrahydropyrimido [4′,5′:4,5]furo[2,3-c]isoquinoline-5,8-diamine

Obtained (12%) from the title compound of Example 19 and cyclobutylamine following the experimental procedure described in Example 3.

LRMS: m/z 451 (M+1)⁺.

### Example 47

### 1-(3-{[2,2-Dimethyl-5-(methylamino)-1,2,3,4-tetrahydropyrimido[4′,5′:4,5]furo[2,3-c]isoquinolin-8-yl]amino}propyl)pyrrolidin-2-one

The title compound of Preparation 33 (15 mg, 0.03 mmol) is suspended in toluene (2 ml) and aluminium trichloride (7.8 mg, 0.06 mmol) is added. This reaction mixture is heated under reflux for 1 h. Once at room temperature, ethyl acetate is added and this organic phase is washed three times with water and then with brine. After drying over magnesium sulfate, the organic phase is filtered and the solvent evaporated under reduced pressure. The reaction crude is passed through a silica-gel column eluting with CH₂Cl₂/MeOH 98:2, to yield 52 mg of the final compound. Yield=49%.

¹H NMR (300 MHz, CHLOROFORM-D) δ ppm 1.1 (s, 6H) 1.7 (t, J=6.6 Hz, 2H) 1.9 (m, 2H) 2.1 (m, 2H) 2.4 (m, 4H) 3.1 (m, J=4.9 Hz, 5H) 3.4 (m, 4H) 3.7 (q, J=6.6 Hz, 2H) 4.7 (m, 1H) 5.8 (t, J=6.6 Hz, 1H) 8.6 (s, 1H).

### Example 48

### N⁵,2,2-trimethyl-N⁸-(2-morpholin-4-ylethyl)-1,4-dihydro-2H-pyrano[4″,3″:4′,5′]pyrido[3′,2′:4,5]furo[3,2-d]pyrimidine-5,8-diamine

The title compound of example 40 (118 mg, 0.2 mmol) is suspended in toluene (30 ml) and aluminium chloride is added (125 mg, 0.9 mmol). This mixture is refluxed for 2 h. The solvent is evaporated under reduced pressure and the residue is partitioned between ethyl acetate and saturated solution of ammonium chloride. After usual work-up, the reaction crude is passed through a silica-gel column, eluting successively with dichloromethane, CH₂Cl₂/MeOH 99:1, CH₂Cl₂/MeOH 98:2 and finally with CH₂Cl₂/MeOH 96:4. 30 mg of the desired final compound are isolated. Yield=31%.

¹H NMR (300 MHz, CHLOROFORM-D) δ ppm 1.4 (s, 6H) 2.6 (s, 4H) 2.7 (s, 2H) 3.2 (s, 3H) 3.2 (s, 2H) 3.3 (s, 2H) 3.8 (s, 5H) 4.3 (m, 1H) 4.6 (s, 2H) 8.6 (s, 1H).

The following examples illustrate pharmaceutical compositions according to the present invention.

### COMPOSITION EXAMPLES

### Composition Example 1

### Preparation of Tablets

### Formulation:

| | |
|---|---|
| Compound of the present invention | 5.0 mg |
| Lactose | 113.6 mg |
| Microcrystalline cellulose | 28.4 mg |
| Light silicic anhydride | 1.5 mg |
| Magnesium stearate | 1.5 mg |

Using a mixer machine, 15 g of the compound of the present invention are mixed with 340.8 g of lactose and 85.2 g of microcrystalline cellulose. The mixture is subjected to compression moulding using a roller compactor to give a flake-like compressed material. The flake-like compressed material is pulverised using a hammer mill, and the pulverised material is screened through a 20 mesh screen. A 4.5 g portion of light silicic anhydride and 4.5 g of magnesium stearate are added to the screened material and mixed. The mixed product is subjected to a tablet making machine equipped with a die/punch system of 7.5 mm in diameter, thereby obtaining 3,000 tablets each having 150 mg in weight.

### Composition Example 2

### Preparation of Coated Tablets

### Formulation:

| | |
|---|---|
| Compound of the present invention | 5.0 mg |
| Lactose | 95.2 mg |
| Corn starch | 40.8 mg |
| Polyvinylpyrrolidone K25 | 7.5 mg |
| Magnesium stearate | 1.5 mg |
| Hydroxypropylcellulose | 2.3 mg |
| Polyethylene glycol 6000 | 0.4 mg |
| Titanium dioxide | 1.1 mg |
| Purified talc | 0.7 mg |

Using a fluidised bed granulating machine, 15 g of the compound of the present invention are mixed with 285.6 g of lactose and 122.4 g of corn starch. Separately, 22.5 g of polyvinylpyrrolidone is dissolved in 127.5 g of water to prepare a binding solution. Using a fluidised bed granulating machine, the binding solution is sprayed on the above mixture to give granulates. A 4.5 g portion of magnesium stearate is added to the obtained granulates and mixed. The obtained mixture is subjected to a tablet making machine equipped with a die/punch biconcave system of 6.5 mm in diameter, thereby obtaining 3,000 tablets, each having 150 mg in weight.

Separately, a coating solution is prepared by suspending 6.9 g of hydroxypropylmethyl-cellulose 2910, 1.2 g of polyethylene glycol 6000, 3.3 g of titanium dioxide and 2.1 g of purified talc in 72.6 g of water. Using a High Coated, the 3,000 tablets prepared above are coated with the coating solution to give film-coated tablets, each having 154.5 mg in weight.

### Composition Example 3

### Preparation of Capsules

### Formulation:

| | |
|---|---|
| Compound of the present invention | 5.0 mg |
| Lactose monohydrate | 200 mg |
| Colloidal silicon dioxide | 2 mg |
| Corn starch | 20 mg |
| Magnesium stearate | 4 mg |

25 g of active compound, 1 Kg of lactose monohydrate, 10 g of colloidal silicon dioxide, 100 g of corn starch and 20 g of magnesium stearate are mixed. The mixture is sieved through a 60 mesh sieve, and then filled into 5,000 gelatine capsules.

### Composition Example 4

### Preparation of a Cream

### Formulation:

| | |
|---|---|
| Compound of the present invention | 1% |
| Cetyl alcohol | 3% |
| Stearyl alcohol | 4% |
| Gliceryl monostearate | 4% |
| Sorbitan monostearate | 0.8% |
| Sorbitan monostearate POE | 0.8% |
| Liquid vaseline | 5% |
| Methylparaben | 0.18% |
| Propylparaben | 0.02% |
| Glycerine | 15% |
| Purified water csp. | 100% |

An oil-in-water emulsion cream is prepared with the ingredients listed above, using conventional methods.

1. A pyridofuropyrimidine derivative of formula (I): 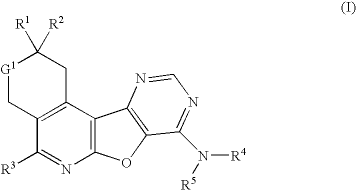
wherein
G¹ is a group chosen from —CR⁶R⁷— and —O— wherein R⁶ and R⁷ are independently chosen from hydrogen atoms and C₁₋₄ alkyl groups;
R¹ and R² are each independently chosen from hydrogen atoms and C₁₋₄ alkyl groups;
R³ is chosen from C₁₋₄ alkyl, C₁₋₄ alkoxy, amino, hydroxy, mono-C₁₋₄alkylamino, di-C₁₋₄alkylamino, C₃₋₈cycloalkylamino, aryl, heteroaryl and saturated N-containing heterocyclyl groups bound to the pyridine ring through their nitrogen atom, wherein each C₁₋₄ alkyl, C₁₋₄ alkoxy, amino, hydroxy, mono-C₁₋₄alkylamino, di-C₁₋₄alkylamino, C₃₋₈cycloalkylamino, aryl, heteroaryl and saturated N-containing heterocyclyl group is optionally substituted by one or more substituents chosen from halogen atoms and hydroxy, C₁₋₄ alkyl, C₁₋₄alkoxy-C₁₋₄alkyl, aryl-C₁₋₄alkyl, −O(CO)OR⁸, C₁₋₄ alkoxy, —(CO)N⁸R⁹, —CN, CF₃, —NR⁸R⁹, —SR⁸ and —SO₂NH₂ groups, wherein R⁸ and R⁹ are each independently chosen from a hydrogen atom or a C₁₋₄ alkyl group;
R⁴ and R⁵ are each independently chosen from hydrogen atoms, C₁₋₄alkyl groups, hydroxyl-C₁₋₄alkyl groups and groups of formula (II): 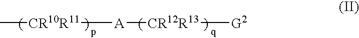
wherein p and q are integers chosen from 0, 1, 2 and 3; A is either a direct bond or a group chosen from —CONR¹⁴—, —NR¹⁴CO—, —O—, —COO—, —OCO—, —S—, —SO— and —SO₂—, wherein each R¹⁰, R¹¹, R¹², R¹³ and R¹⁴ is independently chosen from a hydrogen atom and a C₁₋₄alkyl group and G² is chosen from aryl, heteroaryl and heterocyclyl groups; wherein the group G² is optionally substituted by one or more substituents chosen from halogen atoms and C₁₋₄alkyl, hydroxy, oxo, C₁₋₄alkoxy-C₁₋₄alkyl, aryl-C₁₋₄alkyl, —(CO)OR¹⁶, C₁₋₄alkoxy, —(CO)NR⁶R⁷, —CN, —CF₃, —NR¹⁶R¹⁷, —SR¹⁶ and —SO₂NH₂ groups; wherein R¹⁶ and R¹⁷ are each independently chosen from a hydrogen atom and a C₁₋₄alkyl group; or a pharmaceutically acceptable salt thereof or a N-oxide thereof.

2. The compound according to claim 1, wherein G¹ is chosen from —C(CH₃)₂— and —O—.

3. The compound according to claim 1, wherein both R¹ and R² are methyl groups;

4. The compound according to claim 1 wherein R³ is a group chosen from C₁₋₄ alkyl, C₁₋₄ alkoxy, hydroxy, mono-C₁₋₄alkylamino, di-C₁₋₄alkylamino, C₃₋₈cycloalkylamino, and saturated N-containing heterocyclyl groups which are bound to the pyridine ring through their nitrogen atom, wherein each C₁₋₄ alkyl, C₁₋₄ alkoxy, hydroxy, mono-C₁₋₄alkylamino, di-C₁₋₄ alkylamino, C₃₋₈cycloalkylamino, and saturated N-containing heterocyclyl group is optionally substituted by one or more substituents chosen from halogen atoms and hydroxyl or C₁₋₄ alkyl groups.

5. The compound according to claim 4, wherein R³ is a group chosen from mono-C₁₋₄alkylamino, di-C₁₋₄alkylamino, C₃₋₈cycloalkylamino, and saturated N-containing heterocyclyl groups bound through the nitrogen atom to the pyridine ring, wherein each mono-C₁₋₄alkylamino, di-C₁₋₄alkylamino, C₃₋₈,cycloalkylamino, and saturated N-containing heterocyclyl group is unsubstituted or substituted by one hydroxyl group.

6. The compound according to claim 1, wherein R⁴ is chosen from a hydrogen atom, 2-hydroxyethyl and 2-morpholin-4-yletyhyl groups.

7. The compound according to claim 6, wherein R⁴ is a hydrogen atom.

8. The compound according to claim 1, wherein R⁵ is chosen from a hydrogen atom, hydroxyalkyl groups and groups of formula (II): 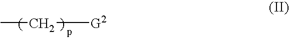
wherein p is an integer chosen from 0, 1, 2 and 3; and G² is a group chosen from aryl, heteroaryl or heterocyclyl groups, wherein each aryl, heteroaryl or heterocyclyl group is optionally substituted with one or more substituents chosen from oxo groups and C₁₋₄alkoxy groups.

9. The compound according to claim 8, wherein G² is chosen from phenyl, pyridine, morpholine and pyrrolidine, wherein each phenyl, pyridine, morpholine and pyrrolidine is optionally substituted with one or more substituents chosen from oxo groups and C₁₋₄alkoxy groups.

10. A pharmaceutical composition comprising a compound according to claim 1 and a pharmaceutically acceptable diluent or carrier.

11. A method for treating a subject afflicted with a pathological condition or disease susceptible to amelioration by inhibition of phosphodiesterase 4, wherein the method comprises administering to the said subject an effective amount of a compound according to claim 1.

12. The method according to claim 11, wherein the pathological condition or disease is chosen from asthma, chronic obstructive pulmonary disease, rheumatoid arthritis, atopic dermatitis, psoriasis and irritable bowel disease.

13. A composition comprising:
(i) a compound as defined in claim 1; and
(ii) another compound chosen from (a) steroids, (b) immunosuppressive agents, (c) T-cell receptor blockers and (d) antiinflammatory drugs.

14. A medicament comprising a compound according to claim 1.

15. (canceled)

16. (canceled)
